(19) United States
(12) Patent Application Publication
(10) Pub. No.: US 2008/0039422 A1
(43) Pub. Date: Feb. 14, 2008

(54) Vitamin B12-Containing Compositions and Methods of Use

(75) Inventors: Antonio Cruz, Toronto (CA); Zdenek Pristupa, Ontario (CA)

Correspondence Address: FISH & RICHARDSON P.C., PO BOX 1022, MINNEAPOLIS, MN, 55440-1022, US

(73) Assignee: Transition Therapeutics Inc., Toronto, ON (CA)

(21) Appl. No.: 11/547,556

(22) PCT Filed: Mar. 30, 2005
(86) PCT No.: PCT/CA05/00478

§ 371 Date: Jan. 9, 2007

Related U.S. Application Data
(60) Provisional application No. 60/558,079, filed on Mar. 30, 2004.
(60) Provisional application No. 60/557,879, filed on Mar. 30, 2004.

(51) Int. Cl. A61K 31/714; A61P 29/00; A61P 3/02

(52) U.S. Cl. 514/52

(57) ABSTRACT

The present invention features compositions that include one or more vitamin B12 compounds and one or more excipients that enhance solubility of the vitamin B12 compounds. In aspects of the invention, the excipients are alcohols, in particular ethanol, propylene glycol, a polyethylene glycol (PEG), glycerol, mannitol, sorbitol, Tween 20, or dimethylsulfoxide or a combination thereof, and/or a salt former. The compositions optionally comprise one or more therapeutic agents other than a vitamin B12 compound. The invention also contemplates processes by which the compositions can be made; kits containing them (or one or more of the components thereof); and methods of using them to treat patients who have a vitamin B12 deficiency, a proliferative disease, an inflammatory disease, or a viral disease.

### FIELD OF THE INVENTION

The present invention provides novel compositions comprising a vitamin B12 compound, processes for preparing the compositions, kits containing them, and methods of treatment and uses with such compositions.

### BACKGROUND OF THE INVENTION

The family of vitamin B12 compounds includes cobalt-containing B complex vitamins that are important for many metabolic processes. For example, these vitamins can function as essential co-factors in the process of DNA synthesis and in the course of cell division. Biochemical evidence suggests that vitamin B12 compounds may up-regulate gene transcription and, thereby, protein synthesis (Watanabe et al., J. Neurological Sci. 122:140-143, 1994).

When food is consumed, digestive enzymes and gastric acid assist in the release of vitamin B12, freeing it to bind to haptocorrin. Pancreatic enzymes subsequently degrade the haptocorrin and release vitamin B12, whereupon it becomes complexed with intrinsic factor (IF). This B12-IF complex binds to receptors on ileal enterocytes and is internalized via endocytosis. Inside the enterocyte, vitamin B12 binds to transcobalamin II (TC II) binding protein and is exported into the portal blood and general circulation. Parenterally administered vitamin B12 bypasses the IF process and vitamin B12 binds directly with free TC II in the plasma. Regardless of the precise route of administration, the vitamin B12-TC II complex interacts with specific TC II receptors on the cell membrane of target cells, and the complex is internalized. Once inside the cells, the TC II-Cb complex is degraded by lysosomes and the cobalamin is metabolized into either methylcobalamin in the cytosol or to adenosylcobalamin in the mitochondria (Seetharam et al., J. Nutr. 129:1761-1764, 1999).

Vitamin B12 is distributed into the liver, bone marrow, and virtually all other tissues including the placenta. This widespread distribution reflects vitamin B12's critical role in cellular reproduction and growth. The liver is the major handling and storage site of vitamin B12. Body concentrations of vitamin B12 increase with age in healthy adults where the total body content is 1-11 mg, with 5 mg on average, of which 50-90% is contained within the liver (Vitamin B12: AHFS Drug Information, American Society of Health-System Pharmacists, Inc., McEvoy G. K., Ed., 2004).

Deficiencies of vitamin B12 can provoke pernicious anemia and such deficiencies are associated with disorders of the nervous system and immune system (Tamura et al., Clin. Exp. Immunol. 116:28-32, 1999; Sakane et al., J. Clin. Immunol. 2:101-109, 1982). More specifically, studies have suggested that vitamin B12 compounds can act as anti-proliferative agents (Nishizawa et al., Intl. J. Vitamin Nutrition Res. 67:164-170, 1997; Shimizu et al., Oncology 44:169-173, 1987; Poydock et al., Exp. and Cellular Biol. 47:210-217, 1979; and Poydock et al., Am. J. Clin. Oncol. 8:266-269, 1985); anti-inflammatory agents (U.S. Pat. No. 5,508,271; U.S. Pat. No. 5,964,224; U.S. Pat. No. 5,716,941); and anti-viral agents (Weinberg et al., Blood 86:1281-1287, 1995; Weinberg et al., Biochem. Biophys. Res. Comm. 246:393-397, 1998; Lott et al., Proc. Natl. Acad. Sci. USA 98:4916-4921, 2001; Poydock, Exp. and Cellular Biol. 47:210-217, 1979; and Tsao et al., Pathobiol. 58:292-296, 1990). Others have suggested that particular vitamin B12 compounds can be used in combination with other therapeutic agents to treat specific inflammatory or proliferative diseases (European Patent No. 0835660; U.S. Pat. No. 6,096,737).

### SUMMARY

The present invention is based, in part, on the discovery of methods of producing compositions that include concentrated amounts of vitamin B12 compounds and one or more excipients. Accordingly, the invention encompasses compositions (e.g., solutions) containing one or more vitamin B12 compounds and one or more excipients, processes by which these compositions are made, kits containing them, and methods of using them to treat patients in need of such treatment (e.g., those who have a vitamin B12 deficiency, cancer (or other unwanted cellular proliferation), an inflammatory disease, or a viral infection).

The present invention relates to novel pharmaceutical compositions of vitamin B12 compounds with one or more selected excipient that enhances solubilization and/or stability of the vitamin B12 compound. The compositions may provide advantageous methods of treatment or protection (e.g. prevention of a disease disclosed herein) of subjects by demonstrating improved chemical, biological, and/or physical properties. Thus, the compositions may address the need for more efficacious dosage forms for vitamin B12 compounds.

In an aspect, the invention provides a pharmaceutical composition comprising a vitamin B12 compound and one or more excipient that enhances or effects solubilization of the vitamin B12 compound (i.e. solubilizing agent), and optionally a pharmaceutically acceptable carrier, vehicle or diluent. In particular, the excipient is selected and/or is in an amount to substantially solubilize a vitamin B12 compound.

Selected compositions according to the present invention include liquids (solutions, syrups, colloids, or emulsions) and lyophilized forms. In an aspect the invention relates to a liquid composition comprising a vitamin B12 compound and a solvent system which enhances or effects solubilization of the vitamin B12 composition. The invention also provides a drug comprising a liquid composition of a vitamin B12 compound and at least one excipient that enhances solubilization of the vitamin B12 compound.

In a composition of the invention, a vitamin B12 compound and excipient or solubilizing agent may be in a ratio selected to augment the solubility or stability of the vitamin B12 compound, augment the activity of the vitamin B12 compound, or provide a beneficial effect.

In aspects of the invention the vitamin B12 compound is cyanocobalamin, adenosylcobalamin, aquocobalamin, methylcobalamin, or 5-o-methylbenzylcobalamin, or an analog or derivative thereof.

In other aspects of the invention the excipient is an alcohol and/or a salt former. In particular aspects the excipient is ethanol, a propylene glycol, a polyethylene glycol (PEG), a glycerol, a sorbitol, a mannitol Tween 20, a dimethylsulfoxide, an organic base, or a combination thereof.

In further aspects of the invention the excipient(s) can constitute about 1-99% and preferably 5-95% (e.g., about 1, 5, 10, 15, 20, 25, 30, 35, 40, 45, 50, 55, 60, 65, 70, 75, 80, 85, 90, 95 or 99%) of the composition. In a particular aspect, 2-5%, 5-10%, 2-10%, 10-20%, 20-30%, 30-40%, 40-50%, 50-60%, 60-70%, or 70-80% of a composition of the invention, by volume is the excipient. This is true whether one uses a single excipient or a combination of excipients. For example, the compositions of the invention can include a concentrated vitamin B12 compound, ethanol, and propylene glycol or PEG. In combination, the ethanol and propylene glycol or PEG can constitute about 5-95% (e.g., about 5, 10, 15, 20, 25, 30, 35, 40, 45, 50, 55, 60, 65, 70, 75, 80, 85, 90, or 95%) of the composition. A composition of the invention may further comprise a salt former such as an organic base.

In other aspects of the invention the concentration of a vitamin B12 compound in a composition of the invention can be at least about 20-500 mg/ml (e.g., at least 20, 25, 30, 35, 40, 45, 50, 55, 60, 65, 70, 75, 80, 85, 90, 95, 100, 125, 150, 200, 225, 250, 275, 300, 325, 350, 375, 400, 425, 450, 475, 500 or more mg/ml).

In a particular aspect, the invention provides a composition of a vitamin B12 compound solubilized in an excipient or solubilizing agent to provide a concentration of a vitamin B12 compound of at least about 30-500 mg/ml, 40-200 mg/ml, or 60 to 200 mg/ml.

In another particular aspect of the invention, a pharmaceutically acceptable composition is provided, which is a solution, comprising a vitamin B12 compound and at least one alcohol, wherein the composition contains at least about 20 mg/ml of the vitamin B12 compound. In another particular aspect, a pharmaceutically acceptable composition is provided, which is a solution, comprising a vitamin B12 compound and at least one excipient, wherein the composition contains at least about 20 mg/ml of the vitamin B12 compound and the excipient is ethanol, propylene glycol, a polyethylene glycol (PEG), glycerol, mannitol, sorbitol, Tween 20, or dimethylsulfoxide or a combination thereof. In a further aspect of the invention, a pharmaceutically acceptable composition is provided, which is a solution, consisting of a vitamin B12 compound, water, and an excipient, wherein the composition contains at least about 20 mg/ml of the vitamin B12 compound.

Compositions of the invention can also be formulated to include other therapeutic agents (i.e., therapeutic agents in addition to a vitamin B12 compound). For example, the vitamin B12-containing compositions of the invention can also include agents such as anti-proliferative (e.g., chemotherapeutic), anti-inflammatory, and/or anti-viral agents. The concentrated vitamin B12 compound may enhance the efficacy of such agents and may, therefore, provide an improved way to treat patients who have been diagnosed as having for example, cancer (or a disease associated with other unwanted cellular proliferation), an inflammatory disease (e.g., an autoimmune disease such as rheumatoid arthritis or multiple sclerosis) or a viral infection (e.g., infection with a human immunodeficiency virus, a hepatitis C virus, or hepatitis B virus).

The invention also relates to a process for preparing a composition of the invention. In an aspect, the invention provides a method for preparing a pharmaceutical composition comprising mixing a vitamin B12 compound and an excipient of the vitamin B12 compound, and optionally a pharmaceutically acceptable carrier, vehicle, or diluent.

A method of increasing solubility of a vitamin B12 compound is contemplated comprising including with the vitamin B12 compound an excipient that enhances or effects solubilization of the vitamin B12 compound. In an aspect, the excipient is an alcohol. In a particular aspect the excipient is one or more of ethanol, propylene glycol, a polyethylene glycol (PEG), a glycerol, sorbitol, mannitol Tween 20, dimethylsulfoxide, or a combination thereof. In a more particular aspect, the excipient is one or more of ethanol, a propylene glycol, a polyethylene glycol.

The methods described herein can also be carried out in the event a composition contains a plurality of components. For example, the methods described herein can be carried out when the composition contains one, two or more vitamin B12 compounds or one, two, or more excipients (e.g., ethanol and propylene glycol or ethanol and PEG). Vitamin B12 containing compositions made by these methods are also within the scope of the present invention.

As there may be advantages to mixing the vitamin B12 compound and the excipient near the time of use, the invention encompasses kits in which the components of the vitamin B12-containing compositions are packaged separately. For example, the kit can contain a vitamin B12 compound in a dry form, typically as a powder, often in a lyophilized form in, for example, a sterile vial or ampule and, in a separate container within the kit, an excipient or a component of an excipient. Any of the vitamin B12 compounds described herein, any of the excipients described herein, and any combination of vitamin B12 compounds and excipients can be included in the kit. In the event the composition intended for administration contains a therapeutic agent in addition to vitamin B12 (e.g., an anti-neoplastic, anti-inflammatory, or anti-viral agent), the kit can also include that therapeutic agent. The “second” therapeutic agent can be combined with the vitamin B12, combined with the excipient, or packaged separately. For example, a kit can contain a vitamin B12-containing composition, or the components thereof, and, in a separate container, an interferon, such as IFNα. Optionally, the kit may also contain instructions for preparation or use (e.g., written instructions printed on the outer container or on a leaflet placed therein) and one or more devices to aid the preparation of the solution and its administration to a patient (e.g., one or a plurality of syringes, needles, filters, tape, tubing (e.g., tubing to facilitate intravenous administration) alcohol swabs and/or Band-Aids®).

In other embodiments, the kits of the invention can include pre-mixed vitamin B12 compounds and instructions for solubilizing any precipitate that may have formed during shipping or storage. Kits containing solutions of one or more vitamin B12 compounds and one or more excipients may also contain any of the materials mentioned above (e.g., any device to aid in preparing the solution for administration or in the administration per se). The instructions in these kits may describe suitable indications (e.g., a description of patients amenable to treatment) and instructions for administering the composition to a patient.

A composition of the invention can be administered to a subject to treat and/or prevent a condition or disease disclosed herein. Therefore, the invention relates to a method for preventing and/or treating a condition or disease disclosed herein comprising administering a therapeutically effective amount of a composition of the invention. Prophylactic and therapeutic methods are also provided comprising administering to a subject in need a therapeutically effective amount of a composition of the invention.

Pharmaceutical compositions of the invention may be adapted for administration to a subject in a number of ways. They may be administered in a convenient manner such as by oral and parenteral (e.g. intravenous, intraperitoneal, intramuscular, intraarticular, intrasternal, injection, infusion, and subcutaneous) routes.

The invention contemplates a method of administering a poorly soluble vitamin B12 compound to a subject in need thereof comprising administering a composition containing the vitamin B12 compounds and at least one excipient or solubilizing agent that enhances solubilization of the vitamin B12 compound.

The invention also contemplates the use of any of the compositions of the invention for preventing, and/or ameliorating disease severity, disease symptoms, and/or periodicity of recurrence of a condition or disease disclosed herein.

The invention relates to the use of a vitamin B12 compound and at least one solubilizing agent or excipient in the preparation of a medicament for treating a condition or disease disclosed herein.

In particular aspects, the compositions of the invention can be used to treat a patient who has a vitamin B12 deficiency (which may result in a neurological deficit), a proliferative disease, an inflammatory disease, or a viral disease. Exemplary and specific disorders are described further below. Those of ordinary skill in the art routinely diagnose such conditions and are well able to identify patients who are likely to benefit from treatment with vitamin B12. The methods can include two steps: (a) identifying a patient in need of treatment and (b) administering, to the patient, a therapeutically effective amount of a vitamin B12-containing composition described herein (e.g., a pharmaceutically acceptable (i.e., non-toxic) composition including cyanocobalamin and ethanol). The patient can be, but is not necessarily, a human patient.

While routes of administration and dosages are described further herein, it is noted that patients can receive at least about 10-5,000 mg of a vitamin B12 compound, formulated as described herein (e.g., at least about 10, 100, 200, 500, 1,000, 2,000, 2,500, 3,000, 3,500, 4,000, 4,500, or 5,000 mg) of the vitamin B12 compound by a parenteral route, such as an intravenous, intramuscular, or subcutaneous route. Such dosages can be divided, and may be given, for example, once, twice or three times daily, weekly, or monthly. The dosages and treatment regimes described herein are applicable to any patient, whether that patient has, or is believed to have, a vitamin B12 deficiency or not; whether that patient has, or is believed to have, a condition associated with a vitamin B12 deficiency or not; or whether that patient has, or is believed to have, any other condition, disease, or disorder.

Given that high concentrations of vitamin B12 compounds can be achieved in the compositions of the present invention, it is expected that physicians (or others) can treat a subject by using (e.g., injecting) a smaller volume of the composition than would otherwise be necessary. This may make the administration less unpleasant for the patient (and may facilitate compliance with a treatment regime). While this advantage may be apparent with at least some embodiments, it is not a required characteristic for the compositions of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

FIGS. 1A-1D are line graphs illustrating the amounts of CN-Cbl in solution with two different combinations of excipients over a short period of time (˜8 hours). The results are presented as the concentration of CN-Cbl (FIGS. 1A and 1C) and as the percentage of CN-Cbl in solution (FIGS. 1B and 1D). The CN-Cbl was added to either 15% ethanol and 20% propylene glycol (FIGS. 1A and 1B) or to 20% ethanol and 40% propylene glycol (FIGS. 1C and 1D) in challenge amounts of: 50 (▪), 75 (▴), 100 (▾), 150 (♦), and 200 (●) mg/ml CN-Cbl.

FIGS. 2A-2D are line graphs illustrating the amounts of CN-Cbl in solution with two different combinations of excipients over an extended period of time (˜200 hours). The results are presented as the concentration of CN-Cbl (FIGS. 2A and 2C) and as the percentage of CN-Cbl in solution (FIGS. 2B and 2D). The CN-Cbl was added to either 15% ethanol and 20% propylene glycol (FIGS. 2A and 2B) or to 20% ethanol and 40% propylene glycol (FIGS. 2C and 2D) in challenge amounts of: 50(▪), 75 (▴), 100 (▾), 150 (♦), and 200 (●) mg/ml CN-Cbl.

FIG. 3 is a graph showing the solubility of cyanocobalamin in various concentrations of choline chloride.

FIG. 4 is a graph showing the results of the filter compatibility study.

### DETAILED DESCRIPTION

### Glossary

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a vitamin B12 compound” includes a mixture of two or more vitamin B12 compounds.

The terms “administering” or “administration” refers to the process by which a therapeutically effective amount of a composition contemplated herein is delivered to a patient for prevention and/or treatment purposes. Compositions are administered in accordance with good medical practices taking into account the patient's clinical condition, the site and method of administration, dosage, patient age, sex, body weight, and other factors known to physicians. “Preventing and/or treating” and “prevention and/or treatment” refer to the administration to a subject of a composition or compounds of a method described herein either before or after onset of a disease. A treatment may be either performed in an acute or chronic way.

Vitamin B12 has been described as having a monovalent cobalt metal surrounded by a porphyrin-like structure of tetrapyrrole rings. A cyano group is bound to the metal center, and a 5′,6′-dimethylbenzimidazolyl nucleotide is linked to the tetrapyrrole rings by a phosphate sugar linkage. A “vitamin B12 compound,” suitable for the present invention includes any of the cobalt-containing B complex vitamins, analogues or derivatives thereof, and conjugates that contain a vitamin B12 compound or an analog or derivative thereof, in particular vitamin B12 or any member of the class of compounds that includes vitamin B12, vitamin B12 analogues, vitamin B12 derivatives, and vitamin B12-containing conjugates. This class includes aquocobalamin, adenosylcobalamin, cyanocobalamin (CN-Cbl), cyanocobalamin carbanalide, hydroxocobalamin (HC), methylcobalamin, nitrosylcobalamin, and 5-o-methylbenzylcobalamin ((5-OmeB-za)CN-Cbl) as well as the desdimethyl, monoethylamide and methylamide analogues of all of the above listed compounds. Also included are the various analogues and homologues of cobamamide, such as coenzyme B12 and 5-deoxydenosylcobalamin. Other analogues include adenosylcyanocobalamin, benzimidazole derivatives such as 5,6-dichlorobenzimidazole, chlorocobalamin, cobalamin lactone, cobalamin lactam, nitrosylcobalamin, sulfitocobalamin, thiocyanatocobalamin, trimethylbenzimidazole, 5-thiocyanatocobalamin, 5-hydroxobenzimidazole, as well as the anilide and ethylamide derivatives of vitamin B12 or its analogues. Other specific derivatives of vitamin B12 include the mono-, di-, and tricarboxylic acid derivatives and the proprionamide derivatives. In addition, the class includes polymers and copolymers of vitamin B12 and any of its analogues or derivatives. Any of the vitamin B12 compounds can be conjugated to at least one other molecule (i.e., to at least one heterologous molecule, including another vitamin B12 compound). The singular form, “Vitamin B12 compound”, may mean any one or more compounds from the class of Vitamin B12 compounds.

Those of ordinary skill in the art will recognize that at least some of the terms used above have synonyms. For example, cyanocobalamin is also known as α-(5,6-dimethyl-benzimidazolyl)cyanocobamide, CN-Cbl, Cy-Cb, and cyanocob(III)alamin.

Although the invention is not limited to compositions that contain vitamin B12 compounds that function by any particular mechanism, it is noted that the biologically active form of the vitamin B12 compound may or may not be the form administered to a subject. The vitamin B12 compound may be a prodrug. For example, many of the vitamin B12 compounds administered may be further processed in vivo. For example, a vitamin B12 compound contained within a solution of the invention (or contained in powder form in a kit of the invention) may be taken up by cells and metabolized into methylcobalamin in the cytosol and/or into adenosylcobalamin in the mitochondria.

Vitamin B12 compounds can be obtained from commercial suppliers (e.g., Sigma-Aldrich Fine Chemicals) or synthesized using methods known in the art.

An excipient for use in the present invention may be selected to improve or enhance the solubility and/or stability of a vitamin B12 compound. In particular an excipient may be selected and/or is in an amount to provide substantial solubilization of a vitamin B12 compound. Substantial solubilization can be to a degree of at least 5, 10, 15, 20, 25, 30, 40, 50, 60, 70, 80, 90, 90-95, 95-99, 95-100%. Substantial solubilization can be essentially complete solubilization (e.g., more than 80%, 90%, 95%, 96%, 97%, 98%, 99%).

In particular, an excipient may be selected to provide the following:
(a) a concentration of a vitamin B12 compound of at least about 20-500 mg/ml (e.g., at least 20, 25, 30, 35, 40, 45, 50, 55, 60, 65, 70, 75, 80, 85, 90, 95, 100, 125, 150, 200, 225, 250, 275, 300, 325, 350, 375, 400, 425, 450, 475, 500 or more mg/ml); and/or
(b) an increase in solubility of a vitamin B12 compound of at least about 2-20 fold, more particularly 2, 5, 10, or 15 fold compared with the solubility of the vitamin B12 compound in water.

An “excipient” can be, or can include, at least one alcohol, which can be monohydric (i.e., an alcohol containing a single hydroxyl (—OH) group); dihydric (i.e., an alcohol containing two hydroxyl groups); trihydric (i.e., an alcohol containing three hydroxyl groups); or polyhydric (“polyols” contain three or more hydroxyl groups). Moreover, the alcohol can be aliphatic (e.g., a paraffinic alcohol, such as ethanol, or olefinic, such as an allyl alcohol); alicyclic (e.g., a cyclohexanol); aromatic (such as phenol and benzyl alcohol); heterocyclic (e.g., furfuryl alcohol); or polycyclic (e.g., a sterol). Dihydric alcohols include glycols and derivatives thereof (diols), and trihydric alcohols include glycerols and derivatives thereof. More specifically, the excipients used in the context of the present invention can be ethanol, propylene glycol (PG), polyethylene glycol (PEG (e.g., PEG 200 or PEG 300)), glycerol, mannitol, sorbitol, Tween 20, or a combination thereof. Other excipients, such as dimethylsulfoxide (DMSO), can also be used alone or in combination with one or more (e.g., two) of the excipients (e.g., one or more of the alcohols) described herein.

An excipient can be, or can include, at least one salt former, including organic bases. Suitable organic bases include without limitation arginine, choline, choline chloride, L-lysine, D-lysine, ornithine, glucamine and its N-mono- or N,N-disubstituted derivatives, benethamine, banzathine, betaine, deanol, diethylamine, 2-(diethylamino)-ethanol, hydrabamine, 4-(2-hydroxyethyl)-morpholine, 1-(2-hydroxyethyl)-pyrrolidine, tromethamine, methylamine, diethanolamine, ethanolamine, ethylenediamine, 1H-imidazole, piperazine, triethanolamine (2,2′,2″-nitrilotris(ethanol), N-methylmorpholine, N-ethylmorpholine, pyridine, dialkylanilines, diisopropylcyclohexylamine, tertiary amines (e.g. triethylamine, trimethylamine) diisopropylethylamine, dicyclohexylamine, N-methyl-D-glutamine, 4-pyrrolidinopyridine, dimethylaminopyridine (DMAP), piperidine, isopropylamine, and meglumine.

The terms “subject” or “patient”, used interchangeably herein, refer to an animal including a warm-blooded animal such as a mammal, which is afflicted with or suspected of having or being pre-disposed to a disease and/or disorder described herein. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. The methods herein for use on subjects/individuals/patients contemplate prophylactic as well as curative use. Typical subjects for treatment include persons susceptible to, suffering from or that have suffered a disease described herein. Subjects may also include persons non-responsive to therapeutic agents (e.g., anti-proliferative, anti-inflammatory, and anti-viral agents) in the absence of a vitamin B12 compound.

“Therapeutically effective amount” relates to the amount or dose of a composition of the invention that will lead to one or more desired beneficial effects (e.g., therapeutic effects). A therapeutically effective amount of compositions of the present invention can vary according to factors such as the disease state, age, sex, and weight of the individual, and the ability of the substance to elicit a desired response in the individual. Dosage regima may be adjusted to provide the optimum therapeutic response (e.g. beneficial effects). For example, several divided doses may be administered daily or the dose may be proportionally reduced as indicated by the exigencies of the therapeutic situation. A therapeutically effective amount may be estimated from cell culture assays or animal cell models.

As used herein, the terms “disorder” and “disease” are used interchangeably to refer to a condition in a subject. The terms include but are not limited to viral, inflammatory, immunodeficiency, and/or proliferative diseases. Certain conditions may be characterized as more than one disease or disorder. For example, certain conditions may be characterized as both proliferative diseases and inflammatory diseases.

“Inflammatory diseases” means a class of diverse diseases and disorders that are characterized by any one of the following: the triggering of an inflammatory response; an upregulation of any member of the inflammatory cascade; the downregulation of any member of the inflammatory cascade. Where a patient is said to have an inflammatory disease, they may have any one of a diverse set of disorders characterized by the influx of certain cell types and mediators that cause tissue damage and, in some cases, death. The inflammatory response is a complex process triggered by an immune response involving chemokines, cytokines, and toxic agents released from activated cells, the up-regulation of cell surface adhesion molecules and trans-endothelial cell migration. Inflammation can occur as a defense to foreign material (such as bacteria or allergens) or to mechanical trauma, toxins, or neoplasia. Autoimmune response by intrinsic stimulation can also induce inflammatory responses.

Inflammatory diseases amenable to treatment with the vitamin B12-containing compositions of the invention include multiple sclerosis, a multi-factorial inflammatory disease of the human central nervous system that results in the slowing of electrical conduction along the nerve. Other diseases amenable to treatment with the compositions of the invention include diabetes (e.g., type I diabetes), artheriosclerosis, inflammatory aortic aneurysm, restenosis, an ischemic or reperfusion injury, glomerulonephritis, sarcoidosis cancer, rheumatic fever, systemic lupus erythematosus, rheumatoid arthritis, Reiter's syndrome, psoriatic arthritis, ankylosing spondylitis, coxarthritis, acute pancreatitis, chronic pancreatitis, an inflammatory bowel disease, ulcerative colitis, Crohn's disease, pelvic inflammatory disease, osteomyelitis, asthma, adult respiratory distress syndrome, wound healing, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. Other inflammatory conditions amenable to treatment occur as a result of a traumatic injury, such as a cut or burn. The singular term “inflammatory disease” includes any one or more diseases selected from the class of inflammatory diseases, and includes any compound or complex disease state wherein a component of the disease state includes a disease selected from the class of inflammatory diseases.

In aspects of the invention, the disease is a neurological disorder. More specifically, neurological disorders that can be treated with the compositions described herein include vacuolar myelopathy, demyelination, spasticity (e.g., spasticity in the legs), encephalopathy, immune mediated encephalitis, subacute encephalitis, calcification of basal ganglia, numbness within an extremity (e.g., within the fingers, hands or forearms), breakdown of myelin and disruption of the axon, pain and tingling in an extremity (e.g., in the feet), distal diminution of sensation, minor motor neuron signs confined to the feet and diminished ankle reflexes, difficulty walking, weakness and uncoordinated legs, distal symmetrical polyneuropathy, inflammatory demyelinating polyneuropathy, multiple neuropathy, progressive polyradiculopathy, autonomic neuropathy, and the like. Conditions of particular interest, which are frequently associated with HIV infection, include vacuolar myelopathy, distal symmetrical polyneuropathy and demyelination.

“Proliferative diseases” means a class of diverse diseases and disorders characterized by a lack of control or poorly controlled cell division or proliferation. Proliferative diseases include disorders associated with an overgrowth of connective tissues, such as various fibrotic conditions, including scleroderma, arthritis, juvenile arthritis, gouty arthritis, and liver cirrhosis, and conditions such as restenosis, arteriosclerosis, and proliferative diabetic retinopathy. Proliferative diseases also include cancer and tumors, such as solid tumors, lymphomas and leukemia, in particular anal cancer, bile duct cancer, colon cancer, esophageal cancer, gallbladder cancer, pancreatic cancer, small intestine cancer, stomach cancer, osteosarcoma, ovarian epithelial cancer, gestational trophoblastic tumor, uterine sarcoma, vaginal cancer, vulvar cancer, ovarian germ cell tumor, soft tissue sarcoma, acute lymphoblastic leukemia, acute myeloid leukemia, small cell lung cancer, malignant mesothelioma, malignant thymoma, hypopharyngeal cancer, laryngeal cancer, nasopharyngeal cancer, oropharyngeal cancer, parathyroid cancer, salivary gland cancer, brain tumor, glioma, cerebellar astrocytoma, cerebral astrocytoma, ependymoma, medulloblastoma, adrenocortical carcinoma, pituitary tumor, islet cell carcinoma, bladder cancer, kidney cancer, penile cancer, Wilm's tumor, AIDS-related lymphoma, cutaneous T-cell lymphoma, Hodgkin's lymphoma, Ewing's sarcoma, chronic myelogenous leukemia, hemangiomas of infancy and childhood, mycosis fungoides, hairy cell leukemia, Kaposi's sarcoma, non-Hodgkin's lymphoma, multiple myeloma, basal cell carcinoma, malignant melanoma, colorectal cancer, non-small cell lung carcinoma, bladder cancer, pancreatic carcinoma, renal cell carcinoma, neuroblastoma, breast cancer, cervical cancer, liver cancer, sarcomas, thyroid cancer, endometrial cancer, uterine cancer, multiple myeloma, testicular cancer, retinoblastoma, oral cancer, rectal cancer, and prostate cancer. The singular form “proliferative disease” includes any one or more diseases selected from the class of proliferative diseases, and includes any compound or complex disease state wherein a component of the disease state includes a disease selected from the class of proliferative diseases.

“Viral diseases” means a class of diverse diseases and disorders caused by or believed to be caused by viruses. The term includes any stage of a viral infection, including incubation phase, latent or dormant phase, acute phase, and development and maintenance of immunity towards a virus. Consequently, the term “treatment” is meant to include aspects of generating or restoring immunity of the patient's immune system, as well as aspects of suppressing or inhibiting viral replication. Viral diseases include, without limitation, genital warts (HPV), HIV/AIDS, herpes, influenza, measles, polio, varicella-zoster, hepatitis A, hepatitis B, hepatitis C, hepatitis D, hepatitis E, hepatitis G., meningitis, genital warts (HPV), a disease associated with papilloma virus infection, a disease associated with influenza virus infection, vesticular stomatitis virus infection, and dengue fever. The singular form “viral disease” includes any one or more diseases selected from the class of viral diseases, and includes any compound or complex disease state wherein a component of the disease state includes a disease selected from the class of viral diseases.

An “anti-inflammatory agent” includes without limitation any member of the classes of compounds used to treat inflammation. The agents include compounds in research, in development and compounds marketed and sold. For example, the vitamin B12-containing compositions of the invention can also include salicylates (e.g., salicin (aspirin), sodium salicylate, choline salicylate, salicylsalicylic acid), diflunisal, salsalate, indomethacin, sulindac, phenylbutazone, oxyphenbutazone, tolmetin, ibuprofen, fenoprofen, flurbiprofen, ketoprofen, mefenamic acid, meclofenamate, piroxicam, naproxen, hydrocortisone, prednisolone, 6-α-methylprednisolone, triamcinolone, dexamethasone, betamethasone, cyclosporine, mycophenolate mofetil, cyclophosphamide, antisense ICAM-1,6-mercaptopurine, tacrolimus, muromonab-CD3, ISAtx247, alefacept, efalizumab, infliximab, azathioprine, methotrexate, fulfasalazine, CT-3™, IPL512,602™, Reliflex™, LFA-1 antagonists, IC74, taxanes, Taxol™, microtubule stabilizing agents, and analogues thereof, glatiramer acetate and analogues thereof, Novantrone™, Antergren™, Campath™, Adapalene™, nitric oxide synthase inhibitors, anti-TN F or IL-1 compounds and antagonists, antibodies to CD52, retinoic acid antagonists, diacerhein and analogues thereof, adhesion peptides, MAF peptides, cytokines, hyaluronic acid binding peptides, RHAMM peptides, integrins, and mixtures thereof. The singular form, “anti-inflammatory compound”, may mean any one or more compounds from the class of anti-inflammatory compounds.

In certain aspects of the invention an anti-inflammatory agent includes non-steroidal anti-inflammatory drugs (NSAIDs), steroidal anti-inflammatory drugs, beta-agonists, anticholingeric agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib, diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib, naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

An “anti-proliferative agent” includes a member of a class of compounds for treating proliferative diseases. Any prophylactic or therapeutic agent which is known to be useful, has been used, or is currently being used for the prevention, treatment, management, or amelioration of one or more symptoms associated with a proliferative disease, such as cancer, can be used in compositions and method of the invention. The agents include compounds in research, in development and compounds marketed and sold. For example, the vitamin B12-containing compositions of the invention can also include altretamine (hexamethylmelamine, Hexylen™), anastrozole (Arimidex™), Exemestane (Aromasin™), bicalutamide (Casodex™), busulfan (Myleran™), capecitabine (Xeloda™), chlorambucil (Leukeran™), cyclophosphamide (Cytoxan™), diethylstilbestrol diphosphate (Stilphostrol™), estramustine (Emcyt™), etoposide (VP-16, Vepesid™), flutamide (Eulexin™), hydroxyurea (Droxia™), Hydrea™, Mylocel™, letozole (Femara™), leucovorin calcium (Leucovorin™), levamisole (Ergamisol™), lomustine (CCNU, CeeNU™), megestrol (Megace™), melphalan (Alkeran™), mercaptopurine (6-MP, Purinethol™), methotrexate (Methotrexate, Rheumatrex™), mitotane (Lysodren™), nilutamide (Nilandron™), procarbazine (Matulane™), tamoxifen (Nolvadex™), testolactone (Teslac™), thioguanine, tretinoin (Vesanoid™), mechlorethamine, 5-fluorouracil, cytarabine, gemcitabine, vinblastine, vincristine, vinorelbine, paclitaxel compound, etoposide, irinotecan, topotecan; leuprolide, flutamide, doxorubicin, bleomycin, epirubicin, mitomycin, interferon compounds (including those described herein), carmustine, lomustine, cisplatin, mitoxantrone (Novantrone™), and mixtures thereof.

In certain aspects the anti-proliferative agent is an immunomodulatory agent such as a chemotherapeutic agent. In other aspects, the anti-proliferative agent is not an immunomodulatory agent. In specific aspects, the anti-proliferative agent is an anti-angiogenic agent. In other aspects, the anti-proiferative agent is not an anti-angiogenic agent.

In embodiments of the invention the anti-proliferative agent is an anti-cancer agent including without limitation acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandomate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin-2 (including recombinant interleukin 2, or rIL2), interferon alpha-2a; interferon alpha-2β; interferon alpha-n1; interferon alpha-n3; interferon beta-I α; interferon gamma-I β; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

In particular aspects of the invention the anti-inflammatory agent and/or proliferative agent is a paclitaxel compound.

“Paclitaxel compound” means paclitaxel and its pro-drugs, analogues, derivatives or conjugates and mixtures thereof. Paclitaxel compounds include but are not limited paclitaxel, TAXOTERE@, TAXOL@, Docetaxel, 10-desacetyl analogues of paclitaxel and TNdesbenzoyl-3′N-t-butoxy carbonyl analogues of paclitaxel, 7-deoxy-docetaxol, 7,8cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10desacetoxytaxol, Protaxol (2′- and/or 7 ester derivatives), (2′- and/or 7 carbonate derivatives), asymmetric synthesis of taxol side chain, fluoro taxols, 9-deoxotaxane, (13acetyl deoxobaccatine III, 9-deoxotaxol, 7-deoxy deoxotaxol, 10-desacetoxy deoxy deoxotaxol, Derivatives containing hydrogen or acetyl group and a hydroxy and tertbutoxycarbonylamino, sulfonated 2′-acryloyltaxol and sulfonated T acyl acid taxol derivatives, succinyltaxol, 2′-γ-aminobutyryltaxol formate, 2′-acetyl taxol, 7-acetyl taxol, 7glycine carbamate taxol, 2′-OH PEG (5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(betaalanyl)-taxol); 2′gamma-aminobutyryltaxol formate; ethylene glycol derivatives of 2′succinyltaxol; 2′-glutaryltaxol; 2′-(N,N-dimethylglycyl) taxol; 21-(2-(N1Ndimethylamino)propionyl)taxol; 2′orthocarboxybenzoyl taxol; Taliphatic carboxylic acid derivatives of taxol, Prodrugs 12′(N,N-diethylaminopropionyl)taxol, 2′(N,Ndimethylglycyl)taxol, 7(N,N-dimethyl glycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7(L-glycyl)taxol, 2′,7-di(L-glycyl)tax.ol, 2′-(L-alanyl)taxol, 7-(L-alanyl)taxol, 2′,7-di(Lalanyl)taxol, 2′-(L-leucyl)taxol, 7-(L-leucyl)taxol, 2′,7-di(L-leucyl)taxol, 2′-(Lisoleucyl)taxol, 7-(L-isoleucyl)taxol, 2′,7-di(L-isoleucyl)taxol, 2′-(L-valyl)taxol, 7-(L14 valyl)taxol, 2′7-di(L-valyl)taxol, 2′-(L-phenylalanyl)taxol, 7-(L-phenylalanyl)taxol, 2′,7-di(Lphenylalanyl)taxol, 2′-(L-prolyl)taxol, 7-(L-prolyl)taxol, 2′,7-di(L-prolyl)taxol, 2′-(Llysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxolj, Taxol analogs with modified phenylisoserine side chains, taxotere, (N-debenzoyl-N-tert(butoxycaronyl) deacetyltaxol, and taxanes (e.g., baccatin M, cephalomannine, 10deacetylbaccatin H1, brevifoliol, yunantaxusin and taxusin). The singular forra, “paclitaxel compound”, means any one or more compounds from the class of paclitaxel. compounds.

An “anti-viral agent” includes without limitation, any member of the classes of compounds used to treat viral diseases. For example, the vitamin B12-containing compositions of the invention can also include interferon compounds (including those described herein), acyclovir, adefovir, abacavir, amprenavir, cidofovir, didanosine, fomivirsen sodium, dipivoxil, adenine, arabinoside, famciclovir, ganciclovir, lopinavir, ritonavir, lamivudine, nelfinavir mesylate, stavudine, trizivir, amivudine, lobucavir, zidovudine, indinavir, nevirapine, delavirdine, saquinavir, efavirenz, ribavirin, foscarnet, n-docosanol, oseltamivir, valacyclovir, palivizumab, doxuridine, miquimod, vidarabine, trifuridine, ritonavir, neuramimidase inhibitor, tenofovir, disoproxil fumarate, zalcitabine, and mixtures thereof.

“Interferon (IFN) compounds” means native sequence interferon polypeptides, isoforms, polypeptide analogues, polypeptide derivatives, chimeric polypeptides, fragments, and variants thereof, or pharmaceutically acceptable salts thereof. In particular, the term refers to interferon-alpha, interferon-alpha analogues, interferon-alpha derivatives, interferon-alpha conjugates, interferon beta, interferon-beta analogues, interferon-beta derivatives, interferon-beta conjugates and mixtures thereof. Naturally occurring interferons can be modified as described here, as can biologically active fragments or other mutants thereof. A fragment, other mutant, analogue, derivative, or IFN-containing conjugate will be “biologically active” so long as it retains sufficient activity to confer a beneficial response in a patient to whom it is administered; it need not retain all, or even substantially all, of the activity of a naturally occurring interferon. Interferon-alpha and interferon-beta genes may be altered by, for example, by oligonucleotide directed mutagenesis to produce interferon-beta analogues thereof, such as the human recombinant cysteine depleted or cysteine replaced analogues. Further, identity or location of more than one amino acid may be changed by targeted mutagenesis. The primary amino acid sequence of the protein may be augmented by glycosylation or joined to supplementary molecules such as lipids, heterologous proteins or peptides, to phosphate groups, and to acetyl groups. Further, individual amino acid residues in the chain may be modified by oxidation, reduction, or other derivatization. The interferon-alpha or interferon-beta protein may be cleaved to obtain fragments which retain activity. The whole protein or its fragments or mutants thereof, can be fused with other peptides and proteins such as immunoglobulins or fragments thereof (e.g. the Fc region of an IgG) and other cytokines by chemical conjugation or by a peptide bond. Interferon-alpha and interferon-beta conjugates may represent, for example, a composition comprising interferon-beta coupled to a non-naturally occurring polymer comprising a polyalkylene glycol moiety.

Examples of interferon compounds include Roferon®, Intron®, Alferong), Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaseron™, Betaferon™, Rebif™, interferon-beta analogues, pegylated interferon-beta, polymerized interferon-beta, dimerized interferon-beta, interferon-beta conjugated to carriers, interferon-beta as oral inhalant, interferon-beta as an injectable composition, interferon-beta as a topical composition, Avonex™analogues, Betaseron™, Betaferon™ analogues, Rebif™ analogues, or biologically active analogues or derivatives thereof. Additionally, agents that induce interferon-alpha or interferon-beta production or mimic the action of interferon-alpha or interferon-beta may also be employed. The singular form, “interferon compound”, may mean any one or more compounds from the class of interferon compounds.

Interferon-alpha may be selected from interferon alpha-2a, interferon alpha-2β, a consensus interferon, a purified interferon alpha product or a pegylated interferon-alpha, including a pegylated interferon-alpha-2a or a pegylated interferon alpha-2 (e.g. Pegasys®). In particular, an interferon-alpha may be selected from interferon alpha-2α, interferon alpha-2β, or a purified interferon alpha product and the amount of interferon-alpha administered may be from 2 to 10 million IU per week on a weekly, TIW, QOD or daily basis. In an embodiment, the interferon-alpha administered is interferon-alpha-2β and the amount of interferon-alpha is administered 3 million IU TIW.

A “native-sequence interferon polypeptide” comprises a polypeptide having the same amino acid sequence of an interferon polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

A “polypeptide analogue” refers to a polypeptide wherein one or more amino acid residues of a native or parent polypeptide have been substituted by another amino acid residue, one or more amino acid residues of a native polypeptide have been inverted, one or more amino acid residues of the native polypeptide have been deleted, and/or one or more amino acid residues have been added to the native polypeptide. Such an addition, substitution, deletion, and/or inversion may be at either of the N-terminal or C-terminal end or within the native polypeptide, or a combination thereof.

Mutations may be introduced into a polypeptide by standard methods, such as site-directed mutagenesis and PCR-mediated mutagenesis. Conservative substitutions can be made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed.

A “polypeptide derivative” refers to a polypeptide in which one or more of the amino acid residues of a native polypeptide have been chemically modified. A chemical modification includes adding chemical moieties, creating new bonds, and removing chemical moieties. A polypeptide may be chemically modified, for example, by alkylation, acylation, glycosylation, pegylation, ester formation, deamidation, or amide formation.

In some aspects of the invention, the interferons used in the vitamin B12-containing compositions of the present invention can be those commercially available as Roferon®, Intron®, Alferon®, Infergen®, Omniferon®, Avonex™, Betaseron™, Betaferon™, Rebif™, or biologically active analogues or derivatives thereof.

In particular applications of the invention, for example where the condition is a neurological disorder including multiple sclerosis, a composition of the invention may additionally comprise interferon (IFN) beta-1β: Betaseron (Berlex [Schering AG]/Chiron), Interferon (IFN) beta-1β: Avonex (Biogen Idec), Chemotherapeutic inhibitor of DNA replication: Novantrone (Amgen/Serono), Recombinant interferon (IFN) beta-1b: Rebif (Serono/Pfizer), copolymer-1 synthetic polymer of four amino acids: Copaxone (Teva), Humanized monoclonal antibody against integrin alpha(4): Tysabri (Biogen Idec/Elan), Oral formulation of cladribine: Milinax (Serono/Ivax), Synthetic myelin basic protein (MBP) peptide: MBP8298 (BioMS Medical), Human MAb against interleukin-12 (IL-12): ABT-874 (Abbott), Oral sustained release 4-aminopyridine (4-AP): Fampridine-SR (Acorda), Immune globulin intravenous (IGIV): Gamunex (Bayer), Oral second generation fumarate (BG12): BG-12 fumarate (Biogen Idec/Fumapharm), AMPA receptor antagonist: E-2007 (Eisai), Dual integrin alpha(4) antagonist: SB683699/T-0047 (GlaxoSmithKline/Tanabe), Humanized MAb against CD52: Campath (Genzyme/Berlex), Vaccine containing mixture of BV5S2, BV6S6 and/or BV13S1 peptides: NeuroVax (Immune Response), HMG-CoA reductase inhibitor: Zocor (Merck), Altered peptide ligand (APL) based on myelin basic protein: NBI-5788 (Neurocrine), Immunomodulator: FTY720 (Novartis), Interferon tau: Tauferon (Pepgen), Humanized anti-CD25 MAb: Zenapax (Protein Design), Oral immune modulating SAIK compound: Laquinimod (SAIK-MS) (Active Biotech/Teva), TNF-alpha inhibitor: Deskar pirfenidone (Marnac), Second-generation antisense targeting integrin alpha(4) (CD49d) mRNA: ATL-1102 (Antisense Therapeutics/Isis), T cell vaccine: Tovaxin (PharmaFrontiers/Opexa), CTLA4-Ig fusion protein: CTLA4-Ig (RG2077) (Repligen), Inhibitor of matrix metalloproteinase-12: MMP-12 inhibitor (Serono), Inhibitor of c-jun N-terminal kinase (JNK): JNK inhibitor (Serono), Oral small molecule inhibitor of integrin alpha(4): CDP323 (UCB), Small molecule inhibitor of leukocyte trafficking: REN-850 (Renovis), and mixtures thereof.

Compositions

The present invention features, inter alia, compositions that include one or more vitamin B12 compounds and one or more excipients and, optionally, one or more therapeutic agents other than vitamin B12; processes by which these compositions can be made; kits containing them (or one or more of the components thereof); and methods of using them to treat patients who have a vitamin B12 deficiency (which may manifest itself as an anemic condition or neurological disorder), or a proliferative disease such as cancer (or other unwanted cellular proliferation), an inflammatory disease (including those that arise in the context of an immune or autoimmune response), or a viral infection.

A vitamin B12 compound and an excipient may be selected to ensure maximum activity and bioavailability of the vitamin B12 compound without increasing any side effects. Compositions of the invention especially include liquid compositions (e.g. solutions, syrups, colloids, or emulsions).

The compositions of the invention may have surprising physiochemical and pharmacological properties. The compositions may have one or more of the following characteristics: favorable solubility, physiological compatible pH, enhanced stability, a long-lasting conservation, a better tolerability, and desirable physical properties (e.g. compression and flow properties) permitting the manufacture of a formulation useful for pharmaceutical medicinal purposes. A vitamin B12 compound in a composition of the invention may be absorbed more rapidly and to a higher degree resulting in improved bioavailability. A composition of the invention may also be substantially non-toxic or have lower toxicity. Accordingly, the compositions of the invention may be very useful as pharmaceutical agents.

A composition of the invention may provide one or more beneficial effect. A beneficial effect can be enhanced biological, physical, and/or chemical properties or augmented desirable therapeutic effects of a vitamin B12 compound. Beneficial effects include but are not limited to increased absorption, distribution, metabolism and/or elimination of the vitamin B12 compound. An excipient employed in the compositions may provide the vitamin B12 compounds in an active form while allowing facile application and administration for particular therapeutic purposes.

The beneficial effects may also be illustrated by increased serum levels of a vitamin B12 compound after administration. A composition of the invention can have increased bioavailability which can be illustrated by an increased rate of dissolution and solubility in comparison to a vitamin B12 compound alone. In an aspect the rate of dissolution (i.e. mass of substance dissolved in a defined time period) of a vitamin B12 compound may be increased up to several fold in a composition of the invention. The solubility (i.e. mass of substance having dissolved clearly in a mass or certain volume of solvent) of a vitamin B12 compound contained in a composition of the invention may be increased. An increase in terminal solubility may result, which is maintained for at least several hours, then decreasing to the solution's degree of saturation.

The vitamin B12-containing compositions can be solutions that include an excipient, which can be, or can include, at least one monohydric, dihydric, trihydric, or polyhydric alcohol which can be aliphatic, alicyclic, aromatic, or polycyclic. More specifically, the excipients used in the context of the present invention can be ethanol, propylene glycol, polyethylene glycol (PEG (e.g., PEG 200 or PEG 300)), glycerol, mannitol, sorbitol, Tween 20, dimethylsulfoxide (DMSO), or a combination thereof. However, a vitamin B12-containing composition that comprises only methylcobalamin and ethanol, or methylcobalamin and DMSO are not contemplated herein.

In aspects of the invention, the excipient is a PEG, in particular PEG 200 or PEG 300, at least 15%, 20%, 30% or 40% ethanol, or propylene glycol, or combinations thereof. In particular aspects the excipient is a combination of propylene glycol and ethanol, more particularly 10-60%, 10-40%, or 20-40% propylene glycol and 5-20% ethanol, most particularly 20-40% propylene glycol and 10%, 15%, or 20% ethanol.

In particular aspects, a composition of the invention comprises a salt former, more particularly an organic base, most particularly choline or choline chloride. The molar ratio of a salt former, in particular choline or choline chloride, to a vitamin B12 compound in a composition of the invention may be about 1:1 to about 1:15 or 1:1 to about 1:10, more particularly about 1:1, 1:3, 1:5 or 1:10. In embodiments of the invention, the amount of salt former, in particular choline or choline chloride, in a composition of the invention is about 5-100 mg/ml, 5-70 mg/ml, 5-50 mg/ml, 5-25 mg/ml, or 5-20 mg/ml.

In specific embodiments, the invention features a pharmaceutically acceptable composition, which is a solution, which includes at least about 20 mg/ml of a vitamin B12 compound (e.g., cyanocobalamin, adenosylcobalamin, aquocobalamin, hydroxocobalamin, methylcobalamin, or 5-o-methylbenzylcobalamin) and at least one alcohol. The vitamin B12 compound can also be an analog or derivative of adenosylcobalamin, aquocobalamin, cyanocobalamin, hydroxocobalamin, methylcobalamin, or 5-o-methylbenzylcobalamin. For example, the analog can be a desdimethyl, monoethylamide, or methylamide analogue. In any of these compositions, the “at least one alcohol” can be ethanol, propylene glycol, a polyethylene glycol (PEG), a glycerol, sorbitol, mannitol or a combination thereof. For example, the pharmaceutically acceptable composition can contain about 20 mg/ml of cyanocobalamin or hydroxocobalamin, ethanol, and propylene glycol or PEG (e.g., PEG 200 or PEG 300). Any of these compositions can also include Tween 20 and/or DMSO. Alternatively, the compositions of the invention can include one or more vitamin B12 compounds and Tween 20 and/or DMSO. For example, the invention features pharmaceutically acceptable compositions (e.g., solutions) that include at least about 20 mg/ml of a vitamin B12 compound (e.g., cyanocobalamin or hydroxocobalamin) and an excipient that includes ethanol, propylene glycol, a polyethylene glycol, glycerol, mannitol, sorbitol, Tween 20, or dimethylsulfoxide.

When present, the amount of the alcohol within a given composition can vary. For example, any of the vitamin B12-containing compounds described herein can be solubilized in a solvent that can include 5-10%, 10-20%, 20-30%, 30-40%, 40-50%, 50-60%, 60-70% or 70-80%, by volume, alcohol (e.g., ethanol) or other excipient. Of course, values within any of these ranges are encompassed as well (e.g., a vitamin B12-containing composition having 10-20% ethanol can include, for example, about 12, 14, 16, or 18% ethanol; a vitamin B12-containing composition having 20-30% ethanol can include, for example, about 22, 24, 26, or 28% ethanol; and so forth).

The compositions of the invention may also be described in terms of the weight of the vitamin B12 compound and the volume of the solution. For example, the compounds of the invention may be described, as noted above, as containing a certain weight of the vitamin B12 compound per volume of solution (e.g., 200 mg/ml). More specifically, solvents used to prepare the compositions of the invention can include 5-10, 10-15, or 15-20% ethanol and 20-30, 30-40, 40-50, or 50-60% propylene glycol or 10-20, 20-30, 30-40, or 40-50% PEG (e.g., PEG 200 or PEG 300) by volume. For example, the compositions can include 5-10, 10-15, or 15-20% ethanol and 60% propylene glycol, each by volume; solutions containing as little as 10% ethanol by volume can improve the solubility of a vitamin B12 compound.

The concentration of the vitamin B12 compound can also vary. For example, the concentration of the vitamin B12 compound, regardless of the excipient (e.g., regardless of the type of alcohol included) can be at least about 25, 30, 35, 40, 45, 50, 55, 60, 65, 70, 75, 80, 85, 90, 95, 100, 125, 150, 175, 200, 225, 250, 275 or 300 mg/ml or more (e.g., at least about 400-500 mg/ml).

In aspects of the invention, compositions of the invention comprise 40-200 mg/ml, more particularly 50-100, 50-195, 50-200, 60-195, 60-200, 70-195, 70-200, 80-195, 80-200, 85-195, 150-195, or 150-180 mg/ml of cyanocobalamin; at least 10%, more particularly 10-25%, 10-20&, 10%, 15%, or 20% ethanol; and, 0-60%, more particularly 0-40%, 5-40%, 20%, 25%, 30%, 35%, or 40% propylene glycol.

Particular compositions of the invention comprise (a) 50-200 mg/ml cyanocobalamin, 15% or 20% ethanol, and 20% or 40% propylene glycol; (b) 50-200 mg/ml, more particularly 50-100 mg/ml cyanocobalamin, 15% ethanol, and 20% propylene glycol; (c) 50-200 mg/ml, more particularly 50-180 mg/ml cyanocobalamin, 20% ethanol, and 40% propylene glycol; (d) 50-200 mg/ml, more particularly 150-195 mg/ml cyanocobalamin, 20% ethanol, and 40% propylene glycol; (e) 50-200 mg/ml, more particularly 50-180 mg/ml cyanocobalamin, 20% ethanol, and 30% propylene glycol; or (f) 50-200, 50-100, 60-100, or 60-80 mg/ml of cyanocobalamin, 10-100 mg/ml, 20-200 mg/ml, 30-80 mg/ml, 40-80 mg/ml, or 50-70 mg/ml choline chloride, 0-60%, 20-40%, 20%, 30%, or 40% propylene glycol and 10-25%, 10-20% %, 10%, 15%, or 20% ethanol.

While methods of administering a vitamin B12-containing composition to a subject are discussed further below, it is noted that particular compositions of the invention encompass those formulated for parenteral administration (e.g., by intramuscular, intravenous, or subcutaneous administration).

A composition of the present invention can also comprise suitable pharmaceutical carriers, vehicles, or diluents selected based on the intended form of administration, and consistent with conventional pharmaceutical practices. Suitable pharmaceutical carriers, vehicles, or diluents are described in the standard text, Remington's Pharmaceutical Sciences (Mack Publishing Company, Easton, Pa., USA 1985). By way of example, suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, targeting agents, and coloring agents may also be combined in the compositions or components thereof. Compositions of the invention can be formulated as neutral or pharmaceutically acceptable salt forms.

A composition of the invention may comprise a unit dosage of at least one vitamin B12 compound and at least one excipient to provide beneficial effects. A “unit dosage” refers to a unitary i.e. a single dose which is capable of being administered to a patient, and which may be readily handled and packed, remaining as a physically and chemically stable unit dose comprising either the active agents as such or a mixture with one or more excipients.

A composition of the invention may be sterilized by, for example, by filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

The vitamin B12-containing compositions of the invention can include other therapeutic agents, such as anti-proliferative (e.g., anti-neoplastic), anti-inflammatory, and/or anti-viral agents. Those of ordinary skill in the art will recognize that some agents can be fairly classified as either an anti-proliferative or anti-viral agent. For example, some viral infections are associated with certain cancers and proliferative disorders; human papilloma viruses are associated with anogenital warts and cervical cancer; hepatitis viruses are associated with liver cancer; the human immunodeficiency virus is associated with Kaposi's sarcoma; etc. Thus, anti-viral agents administered to treat such viral infections may also be acting as anti-proliferative agents.

Similarly, various viral diseases, such as hepatitis B and hepatitis C, as well as various inflammatory disorders, such as multiple sclerosis, can be treated with interferons. For example, a vitamin B12-containing composition of the invention can be administered with an interferon. As with any combination therapy described herein, one can administer the interferon by the same or a different route than the vitamin B12, and at the same or a different time (e.g., minutes or hours later). Alternatively, the interferon and the vitamin B12 can be administered together. In other embodiments, a vitamin B12-containing solution and interferon can be administered with another agent (a third agent), such as an antiviral agent (e.g., ribavirin). As noted, one can administer the vitamin B12, the interferon, and the third agent (e.g., ribavirin) by the same or a different route or at the same or a different time. If desired (e.g., to treat a patient who has hepatitis C), the agents can be combined and administered together. Accordingly, any of the vitamin B12-containing compositions of the invention can include one or more interferons or be administered in conjunction with administration of an interferon. Alternatively, or in addition to the IFNs described above, agents that induce IFN-α or IFN-β production or that mimic the action of IFN-α or IFN-β may also be employed.

Processes for Making a Vitamin B12-Containing Composition.

While aspects of the invention for making vitamin B12-containing compositions are described below, the methods of the invention encompass mixing a vitamin B12 compound (e.g., CN-Cbl, HC, or methylcobalamin) with one or more excipients (e.g., an alcohol, such as ethanol, and propylene glycol) under conditions (e.g., for a time and at a sufficient temperature). The amount of the vitamin B12 compound added to the excipient(s) can vary, as can the precise methods by which the excipient and the vitamin B12 compound are combined. The excipients and/or conditions may be selected to solubilize or substantially or essentially completely solubilize the vitamin B12 compound. In an aspect, the excipient improves the vitamin's solubility. For example, if a given vitamin B12 compound, dissolved in water, reaches a concentration of 15 mg/ml, and the same vitamin B12 compound, dissolved under the same conditions in 90% water and 10% ethanol, reaches a concentration of 30 mg/ml, then 10% ethanol is an amount sufficient to increase the solubility of the vitamin B12 compound. As the experimental results herein illustrate, compositions comprising about 90% water, about 10% alcohol (e.g., 10% ethanol) and about 30 mg/ml of a vitamin B12 compound, are within the scope of the present invention. More specifically, the excipient can constitute about 1-99% and preferably 5-95% (e.g., about 1, 5, 10, 15, 20, 25, 30, 35, 40, 45, 50, 55, 60, 65, 70, 75, 80, 85, 90, 95 or 99%) of the composition. This is true whether one uses a single excipient or a combination of excipients. For example, the compositions of the invention can include a concentrated vitamin B12 compound, ethanol, and propylene glycol or PEG. In combination, the ethanol and propylene glycol or PEG can constitute about 5-95% (e.g., about 5, 10, 15, 20, 25, 30, 35, 40, 45, 50, 55, 60, 65, 70, 75, 80, 85, 90, or 95%) of the composition. The concentration of the vitamin B12 compound can be at least about 20-500 mg/ml (e.g., at least 20, 30, 40, 50, 75, 100, 125, 150, 200, 250, 300, 400, 500 or more mg/ml).

The methods by which the vitamin B12 compounds and the excipients are combined can be carried out in any manner that allows the vitamin B12 compound to dissolve in the excipient. For example, when these two components are combined, one can agitate the combination (by hand or using a device such as a shaker or vortex) and, optionally, apply heat (e.g., the container containing the combined ingredients can be placed in a water bath or incubator set at between about 37° C. and about 65° C. for a period of time ranging from minutes to hours (e.g., 2-5 hours)). If necessary, the combined ingredients can be alternatively shaken and heated. For example, the vitamin B12 compound and a solution containing an excipient can be combined, shaken for 30 seconds, placed in a water bath, and then removed from the bath every 10 minutes or so to be shaken again. Following the incubation, the container can be cooled to room temperature and clarified by centrifugation (e.g., centrifuged for 2-10 minutes (e.g., five minutes) at a relative centrifugal force (rcf) of 16,100). If desired, the concentration of the vitamin B12 compound in the composition can then be determined (by, for example, spectroscopy). To help protect the vitamin B12 compound from degradation, the process can be carried out under conditions where the compound is exposed to light as little as possible or practical. It is expected that the reagents used will be sterile, however the composition can be sterilized or further sterilized (e.g., by autoclaving or filter-sterilization).

The methods described herein can also be carried out in the event a composition contains a plurality of components. For example, the methods described herein can be carried out when the composition contains one, two or more vitamin B12 compounds or one, two, or more excipients (e.g., ethanol and propylene glycol or ethanol and PEG). Vitamin B12 containing compositions made by these methods are also within the scope of the present invention.

Vitamin B12-containing compositions (e.g., solutions) can be made by (a) providing a vitamin B12 compound in an amount described herein (e.g., in an amount sufficient to generate a composition containing at least about 20 mg/ml and up to 5,000 mg/ml), (b) providing an aqueous solution comprising at least one of the excipients described herein (e.g., an alcohol), and (c) generating a mixture of the vitamin B12 compound and the excipient having a volume such that the concentration of the vitamin B12 compound in the mixture is at least about 20 mg/ml. Mixing alone (e.g., simply combining the components) may be sufficient to produce the composition (e.g., the solution). If necessary or desired, the method can further include shaking the mixture (optionally with the aid of a vortex or other mechanical device); and heating the mixture. The mixture can be heated by being placed in a water bath or other temperature-controlled environment at 37-65° C. (e.g., 37° C., 42° C., 50° C., 55° C., 60° C., or 65° C.) for a time sufficient to facilitate solubilization. The steps in which the mixture is shaken and heated can be repeated as many times as necessary until the vitamin B12 compound is solubilized in the solution comprising the excipient. In one embodiment, the shaking can include alternatively vortexing the mixture for about 15-90 seconds and heating the mixture to 45-65° C. Regardless of the dosage intended for administration, more concentrated solutions can be manufactured and diluted at some point prior to administration.

While it is preferable that the amount of the vitamin B12 compound and the nature of the excipient be such that the vitamin B12 compound remains “in solution” for an extended period of time, the invention is not so limited. The compositions of the invention encompass solutions that acquire precipitate over periods of time as short as a few hours or a few days so long as the precipitate can be dissolved prior to administration to a patient or other use.

Where a vitamin B12-containing composition includes another biologically active agent (e.g., one or more anti-proliferative, anti-inflammatory, or anti-viral agents described herein), that agent can be added to the excipient before, at the same time as, or after the vitamin B12 compound is added to the excipient. The amount added to the solution can be the same as the amount used when administered alone. While the invention is not so limited, co-administration of at least some vitamin B12 compounds with at least some of the anti-proliferative, anti-inflammatory, or anti-viral agents described herein may increase the efficacy of the latter agent, thus allowing less concentrated formulations to be made and/or lower dosages to be effectively administered. As noted above, co-administration does not necessarily mean that the biologically active agents are packaged or administered to a patient as a mixture; they can be packaged and administered separately. Kits.

As there may be advantages to mixing the vitamin B12 compound and the excipient near the time of use, the invention encompasses kits in which the components of the compositions are packaged separately. For example, the kit can contain a vitamin B12 compound in a powdered or other dry form in, for example, a sterile vial or ampule and, in a separate container within the kit, an excipient or a component of an excipient (in liquid or dry form). In an aspect, the kit can contain a vitamin B12 compound in a dry form, typically as a powder, often in a lyophilized form in, for example, a sterile vial or ampule and, in a separate container within the kit, an excipient or a component of an excipient. Alternatively, the kit may contain a vitamin B12 compound in the form of a concentrated solution that is diluted prior to administration. Any of the vitamin B12 compounds described herein, any of the excipients described herein, and any combination of vitamin B12 compounds and excipients can be included in the kit. In the event the solution intended for administration contains a therapeutic agent in addition to vitamin B12 (e.g., an anti-proliferative (e.g., anti-neoplastic), anti-inflammatory, or anti-viral agent), the kit can also include that therapeutic agent (in any sufficiently stable form). Such therapeutic agents can be combined with the vitamin B12 compound, combined with the excipient, or packaged separately. For example, a kit can contain a vitamin B12-containing solution, or the components thereof, and, in a separate container, an interferon, such as IFNα. Optionally, the kit may also contain instructions for preparation or use (e.g., written instructions printed on the outer container or on a leaflet placed therein) and one or more devices to aid the preparation of the solution and/or its administration to a patient (e.g., one or a plurality of syringes, needles, filters, tape, tubing (e.g., tubing to facilitate intravenous administration) alcohol swabs and/or Band-Aids®). As noted above, compositions that are more concentrated than those administered to a subject can be prepared. Accordingly, such compositions can be included in the kits of the invention with, optionally, suitable materials (e.g., water, saline, or other physiologically acceptable solutions) for dilution. Instructions included with the kit can include, where appropriate, instructions for dilution.

In other embodiments, the kits of the invention can include pre-mixed vitamin B12 compositions (with or without an additional therapeutic agent) and instructions for solubilizing any precipitate that may have formed during shipping or storage. Kits containing solutions of one or more vitamin B12 compounds and one or more excipients may also contain any of the materials mentioned above (e.g., any device to aid in preparing the composition for administration or in the administration per se). The instructions in these kits may describe suitable indications (e.g., a description of patients amenable to treatment) and instructions for administering the solution to a patient.

Methods of Treatment.

The invention contemplates the use of a composition of the invention for preventing and/or treating a disease or disorder, in particular preventing, and/or ameliorating disease severity, disease symptoms, and/or periodicity of recurrence of a disease or disorder disclosed herein. The invention also contemplates treating in mammals diseases and/or disorders using the compositions or treatments of the invention. The present invention in an embodiment provides a composition comprising a compound that achieves greater solubility and stability.

The invention additionally provides use of at least one vitamin B12 compound and at least one excipient or a composition of the invention in the preparation of medicaments for the prevention and/or treatment of a disease or disorder contemplated herein.

Compounds and compositions of the present invention can be administered by any means that produce contact of the active agent(s) with the agent's sites of action in the body of a subject or patient to produce a beneficial effect. Active ingredients can be administered simultaneously or sequentially and in any order at different points in time, to provide the desired beneficial effects. A composition of the invention can be formulated for sustained release, for delivery locally or systemically. It lies within the capability of a skilled physician or veterinarian to select a form and route of administration that optimizes the effects of the compositions and treatments of the present invention to provide beneficial effects.

The compounds or compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms. The compounds or compositions of the invention may be administered by intranasal route via topical use of suitable intranasal vehicles, or via a transdermal route, for example using conventional transdermal skin patches.

As noted above, vitamin B12 is required for various biological processes, and vitamin B12 deficiencies are known to provoke pernicious anemia and neurological disorders. Accordingly, patients who suffer from a vitamin B12 deficiency, regardless of its cause or the manifest symptoms, are amenable to treatment with the compositions described herein, as are “patients” who have normal circulating levels of vitamin B12. More specifically, the methods of the invention encompass treating (by, e.g., reducing or eliminating) or preventing a vitamin B12 deficiency in a patient by administering, to the patient, a therapeutically effective amount of a pharmaceutically acceptable composition of the present invention. The patient can be identified as one having a less than average circulating level of vitamin B12 or a condition that results therefrom (e.g., pernicious anemia or a neurological disorder).

Patients having any type of proliferative disease or disorder can also be treated with the compositions described herein (whether the unwanted cellular proliferation is malignant or benign; the proliferative disease can be any within the class of diverse diseases and disorders characterized by a lack of control of (or poorly controlled) cell division or proliferation). Thus, the invention provides a method for preventing and/or treating a proliferative disease comprising administering a therapeutically effective amount of a composition of the invention.

The compositions of the invention may also be used to prevent or treat an inflammatory disease, in particular a neurological inflammatory disease including multiple sclerosis. Thus, the invention provides a method of preventing and/or treating an inflammatory disease comprising administering a therapeutically effective amount of a composition of the invention

As noted above, a patient can have a viral disease, including an HIV-related disease, hepatitis B, hepatitis C, a disease associated with papillomavirus infection, or a disease associated with influenza virus infection. Thus, the invention provides a method to prevent or treat a viral disease, in particular hepatitis B or hepatitis C, comprising administering a therapeutically effective amount of a composition of the invention.

Although various disorders are referred to herein as, for example, “neurological” or “inflammatory” or “autoimmune,” or “viral,” each of those terms covers a number of specific diseases, many of which could be fairly placed in more than one category. Moreover, a disease that first presents in one of these categories may progress to a disease that is more frequently assigned to a distinct category. For example, viral infections can result in inflammation and can lead to cancer. The cellular injury that occurs in the course of a viral disease is often the result of an immune reaction against the virus; the consequences of a viral disease depend on both the virus and the host (e.g., the number of infecting viral particles, the speed of viral multiplication, the cellular response to infection, and the host's secondary responses to the cellular injury). Depending on factors such as these, the symptoms of a viral disease can vary widely. For example, an infected individual may experience an asymptomatic infection, an acute clinical disease, a chronic illness, or develop cancer. Viral hepatitis often results in chronic or acute inflammation of the liver and, in some cases, can lead to hepatocellular carcinoma. Neurological dysfunction may occur in association with an immunological disorder such as AIDS or ARC, and can be manifest as depressed ankle reflexes, distal weakness, loss of position and vibratory sense, spasticity, a “pins and needles” sensation, ataxia, or muscle weakness. The vitamin B12-containing compositions described herein can be administered to a patient experiencing any of these symptoms as well as to patients who are otherwise immunocompromised, such as patients taking an immunosuppressant agent.

As noted above, the compositions of the invention can include not only a vitamin B12 compound, but also an additional therapeutic agent. These compositions can be administered to patients who would benefit from receipt of the additional therapeutic agent. For example, compositions containing vitamin B12 and an anti-proliferative (e.g., anti-neoplastic) agent can be administered to treat a patient who has cancer (or other proliferative disorder); compositions containing vitamin B12 and an anti-inflammatory agent can be administered to treat a patient who has a disease in which inflammation is a factor; compositions containing vitamin B12 and an anti-viral agent can be administered to treat a patient who has a viral infection; and so forth.

A vitamin B12 compound may have a synergistic effect with an additional therapeutic agent. It may reduce the amount of additional therapeutic agent needed to be effective, especially in cases where an increased dose of the therapeutic agent is needed due to development of tolerance or other factors after prolonged use of the agent.

Dosage

The subject compounds are administered in amounts sufficient to stabilize or reverse the dysfunction observed (e.g., the neurological deficit). In aspects of the invention a composition of the invention is administered in a therapeutically effective amount. In some embodiments, the dose may be sufficient to upregulate the immune system. This upregulation may be manifested as an increase in the number of T4 and/or T8 positive lymphocytes in a patient (e.g., an HIV-infected patient), as an increase in anti-tumor or other T cell-mediated activity, as an increase in suppresser cells which effect a reduction in autoimmune activity, and the like.

The subject compositions will generally be administered (e.g., parenterally administered) from as often as one or more times daily for initial treatment, to as infrequently as monthly for maintenance level treatment. The amount of the vitamin B12 compound administered may vary with the general health of the patient, the response of the patient to the treatment, whether treatment is being made in combination with other drugs, and the like. More specifically, administration may be one or more times daily, usually not more than about four times, particularly depending upon the level of drug that is administered.

It is noted that patients can receive at least about 10-5,000 mg of a vitamin B12 compound, formulated as described herein (e.g., at least about 10, 100, 200, 500, 1,000, 2,000, 2,500, 3,000, 3,500, 4,000, 4,500, or 5,000 mg) of the vitamin B12 compound by a parenteral route, such as an intravenous, intramuscular, or subcutaneous route. Such dosages can be divided, and may be given, for example, once, twice or three times daily, weekly, or monthly. The dosages and treatment regimes described herein are applicable to any patient, whether that patient has, or is believed to have, a vitamin B12 deficiency or not; whether that patient has, or is believed to have, a condition associated with a vitamin B12 deficiency or not; or whether that patient has, or is believed to have, any other condition, disease, or disorder.

Pharmacokinetic studies have shown that to sustain an elevated circulating amount of vitamin B12 (e.g., greater than about 0.1 μg/ml) it is preferred to administer a vitamin B12-containing solution, by infusion, over time. With the compositions of the present invention, however, it will be possible to achieve the desired result with a bolus injection. Accordingly, the invention encompasses methods of achieving a circulating amount (as measured, for example, in plasma) of a vitamin B12 compound in a subject by administering a solution described herein by bolus injection (e.g., an intravenous or intramuscular injection). The injection can be repeated, of course, as necessary to maintain a desired circulating amount of vitamin B12 (e.g., about 0.1 μg/ml to about 250 μg/ml (e.g., about 0.1-1.0, about 1.0-10.0, about 10-50, about 50-100, about 100-150, about 150-200, about 200-250, about 0.5-200, about 1.0-175, about 5-150, about 15-100, or about 25-75 μg/ml)).

### EXAMPLES

### Example 1

### Cyanocobalamin-Containing Solutions

The experiments conducted in this series feature cyanocobalamin (CN-Cbl). Varying amounts of CN-Cbl were added to various excipients, including water alone, water with one other agent (e.g., ethanol) and water with two other agents (e.g., ethanol and propylene glycol). The stability was tested after varying amounts of time, and following procedures in which the vitamin B12 compound and its excipient were combined by vigorous mixing and heated.

More specifically, for each excipient condition, CN-Cbl was weighed into an Eppendorf tube, and an appropriate volume of excipient solution was added to achieve the “challenge” concentration in a total volume of ˜0.4-0.7 ml. The tube was then vortexed before being heated to 60° C. for two hours, with additional mixing by vortexing carried out for 5-10 seconds every 5-10 minutes.

To study the effect of temperature, in some experiments, the solutions were heated to only 37° C. for 15 minutes (unless otherwise noted, the solutions were prepared as described above: by heating them to 60° C. for two hours, with additional mixing by vortexing carried out for 5-10 seconds every 5-10 minutes).

Following the mixing and heating procedure described above, the tubes were cooled to room temperature (RT) and left at room temperature for 1-2 hours. Before measuring the concentration of CN-Cbl, the solutions were clarified by centrifugation for ten minutes at rcf 16,100. The concentrations of CN-Cbl in the solutions were measured using a spectrophotometer (λ 361 nm; ε 28,100 M⁻¹ cm⁻¹) after being suitably diluted to ensure accurate absorbance measurements in the range of linearity (˜0.20 to 0.80). Cyanocobalamin and cyanocobalamin-containing solutions were protected from light (by being kept covered with foil) as much as practicable.

To determine the stability of the various solutions, the clarified stocks were aliquoted into fresh tubes and stored at room temperature, protected from light, for up to two weeks. Prior to measuring the concentrations of CN-Cbl on subsequent occasions, the tubes were centrifuged for ten minutes at rcf 16,100. The concentration of CN-Cbl was measured as described above by spectrophotometry.

In one study, CN-Cbl was combined with excipients containing propylene glycol, ethanol, PEG 300, or glycerol so that the final maximum concentration of CN-Cbl could be as high as 100 mg/ml. The amount of each excipient was also varied from 5-80% (however, not all possible variants were tested). Table 1 shows the concentration of cyanocobalamin (CN-Cbl; mg/ml) in water and in four excipients: propylene glycol, ethanol, PEG 300, and glycerol. The concentration of CN-Cbl to be solubilized (the “challenge”) in each case was 100 mg/ml of CN-Cbl, and the concentration of each of the excipients varied from 5% to 80% in water. When dissolved in water, the concentration of CN-Cbl reached only 14.1 mg/ml. However, inclusion of glycerol at concentrations of 5, 10, 15, or 20% resulted in concentrations of CN-Cbl of approximately 16, 18, 19, and 21 mg/ml, respectively. The concentrations of CN-Cbl in other excipients were even greater. For example, the concentration of CN-Cbl in solutions containing 40 or 50% PEG 300 was about 45 mg/ml; the concentration in 15% ethanol was about 35 mg/ml; the concentration in 20% ethanol was about 52 mg/ml; and the concentrations in propylene glycol at any concentration over 30% was more than 85 mg/ml (see Table 1).

In other studies, combinations were tested of ethanol and propylene glycol or of ethanol and PEG 300. Tables 2A-2D are tables showing the concentrations of cyanocobalamin (CN-Cbl; mg/ml) in combinations of excipients. Table 2A shows the results obtained when a challenge of 200 mg/ml of CN-Cbl was added to excipients containing various concentrations of ethanol and propylene glycol. The concentrations of ethanol ranged from 0-20% and the concentrations of propylene glycol ranged from 0 to 60%. Table 2B shows the results obtained when a challenge of 200 mg/ml of CN-Cbl was added to excipients containing various concentrations of ethanol and PEG 300. In Table 2C, the amount of propylene glycol and the amount of CN-Cbl (the “challenge”) varied. The excipient contained 20% ethanol and 0-40% propylene glycol, and the amount of CN-Cbl challenge added varied from 50-200 mg/ml. Table 2D reports final concentrations of CN-Cbl when excipients containing varying amounts of propylene glycol and ethanol were challenged with 400 mg/ml CN-Cbl.

In control experiments (i.e., experiments in which CN-Cbl was dissolved in water), the concentration of CN-Cbl in solutions challenged with 200 mg/ml was only about 20 mg/ml. However, when the same “challenge” amount of CN-Cbl was added to solutions having the stated combination of excipients, the final concentration was higher, and in some cases it was much higher, than that achieved with water alone. For example, solutions containing 40% propylene glycol and 15% ethanol contained about 193 mg/ml CN-Cbl. In fact, CN-Cbl dissolved about as well in solutions containing 40-60% propylene glycol and 10, 15, or 20% ethanol (Table 2A). The final concentration of CN-Cbl was not as great when the challenge amount was higher. In experiments where solutions containing 20-40% propylene glycol and 5-20% ethanol were challenged with 400 mg/ml of CN-Cbl, the highest concentration of CN-Cbl achieved was about 115 mg/ml (Table 2D). Therefore, the amount of the challenge may affect the final concentration significantly.

As a significant increase in solubility was observed when the concentration of ethanol was increased from 15% to 20% (see Tables 1 and 2A), and a relatively greater solubility was observed with less aggressive challenge concentrations, a study was conducted in which solutions containing 20% ethanol and 0-40% propylene glycol were challenged with 50-200 mg/ml CN-Cbl. The results are presented in Table 2C. Regardless of the amount of the challenge, no more than 30 mg/ml CN-Cbl was solubilized in water. In contrast, the amount of CN-Cbl solubilized in either 20% ethanol alone or in an excipient containing 20% ethanol and 10-40% propylene glycol was nearly as great as the amount of the challenge material added in nearly all circumstances. For example, the final concentration of CN-Cbl was about 50 mg/ml when 50 mg/ml CN-Cbl was dissolved in solutions containing 20% ethanol and 0-40% propylene glycol. Similarly, the final concentration of CN-Cbl was about 100 mg/ml when 100 mg/ml CN-Cbl was dissolved in the same solutions (i.e., solutions containing 20% ethanol and 0-40% propylene glycol. At higher challenge values, the combination of excipients appeared to be more important. For example, when a solution containing 20% ethanol (without propylene glycol) was challenged with 175 mg/ml CN-Cbl, the final concentration of CN-Cbl was only about 77 mg/ml. However, when the solution contained 20% ethanol and 10-40% propylene glycol, the final concentration of CN-Cbl was much closer to the challenge value [the final concentration of CN-Cbl in solutions containing 20% ethanol and 10% CN-Cbl was about 162 mg/ml; in solutions containing 20% ethanol and 20% propylene glycol CN-Cbl was about 177 mg/ml (essentially the entire challenge volume dissolved); in solutions containing 20% ethanol and 30% propylene glycol CN-Cbl was about 169 mg/ml; and in solutions containing 20% ethanol and 40% propylene glycol CN-Cbl was about 159 mg/ml].

In the experiments described above, the concentration of CN-Cbl was measured within an hour or two after the components of the solutions were initially combined. To determine the stability of the preparations, the concentrations of CN-Cbl were measured in individual excipients after three days and the concentrations of CN-Cbl in combined excipients were measured after two weeks. Tables 3A-3B are tables showing the concentrations of CN-Cbl (mg/ml) in various solutions after three days incubation at room temperature. Solutions containing 0-60% propylene glycol (Table 3A) and solutions containing 0-40% ethanol (Table 3B) were challenged with 50, 75, 100, and 125 mg/ml CN-Cbl. Table 4 is a table showing the concentrations of CN-Cbl in excipients containing ethanol and propylene glycol after two weeks incubation at room temperature. The solutions were challenged with either 50 or 75 mg/ml CN-Cbl and the excipients contained ethanol (10, 15, or 20%) and propylene glycol (20, 30, or 40%). Generally, in these preliminary experiments, the solutions containing combined excipients appeared to be more stable (solutions containing ethanol and propylene glycol were tested further; see below). Regardless of the diminution in concentration over time, however, it was hypothesized that the initial concentrations of CN-Cbl could be restored with an additional application of energy (e.g., mixing, heating, or both mixing and heating; see below).

The effect of heating the solutions to different temperatures on long-term stability was tested. Solutions challenged with 50 or 75 mg/ml CN-Cbl containing 20% ethanol and 20-40% propylene glycol that were prepared at 60° C. and 37° C. were compared. The concentrations of CN-Cbl in solutions prepared at 60° C. were measured after three days, and the concentrations of CN-Cbl in solutions prepared at 37° C. were measured after two weeks. No significant difference was found in the concentrations of CN-Cbl in these solutions when the challenge amount was 50 mg/ml and only a slight difference when the challenge amount was 75 mg/ml. Table 5 is a table showing the effect of heating the solutions on long-term stability. Solutions challenged with either 50 or 75 mg/ml CN-Cbl and containing ethanol and/or propylene glycol were heated to 37° C. (in which case, the concentration of CN-Cbl was measured after two weeks incubation at room temperature) or 60° C. (in which case, the concentration of CN-Cbl was measured after three days incubation at room temperature). Additional information regarding stability was obtained in the experiments described below.

The effect of reheating the samples and establishing more rigorous time courses of stability were tested. To test the effect of reheating, three samples were prepared containing various challenge amounts of CN-Cbl in solutions containing ethanol and propylene glycol: (A) a 100 mg/ml challenge in 15% ethanol, 20% propylene glycol; (B) a 150 mg/ml challenge in 20% ethanol, 40% propylene glycol; and (C) a 200 mg/ml challenge in 20% ethanol, 40% propylene glycol. On Day 1 the samples were heated to 60° C. for three minutes, with vigorous shaking. Each sample was divided into four aliquots. One sample in each of the three groups was cooled to room temperature, centrifuged, and the concentration of the solution therein was measured. On Day 4, the same aliquot was centrifuged and the concentration was measured again. With the three remaining aliquots, concentrations were measured after (1) no reheating; (2) heating to 60° C. for two hours; and (3) heating to 50° C. for two hours.

The samples heated to 60° C. appeared clear. Those heated to 50° C. contained some visible precipitate, and the samples that were not reheated contained significant visible precipitate (see Table 6).

Based on these data, it was concluded that reheating the solutions to 60° C. completely dissolved any CN-Cbl that precipitated in solutions A and B, and nearly dissolved the CN-Cbl that precipitated in solution C. Reheating to 50° C. is partially effective, but not as effective, as heating the solutions to 60° C. Reheating the solutions to 37° C. is not sufficient to redissolve the solutions tested.

To establish more precise time courses, ten samples were prepared containing various challenge amounts of CN-Cbl in solutions containing ethanol and propylene glycol. Five of the solutions contained 15% ethanol and 20% propylene glycol and five contained 20% ethanol and 40% propylene glycol. Within each set of five, they were challenged with: (1) 50 mg/ml CN-Cbl; (2) 75 mg/ml CN-Cbl; (3) 100 mg/ml CN-Cbl; (4) 150 mg/ml CN-Cbl; and (5) 200 mg/ml CN-Cbl. To generate the solutions, the methods described above were modified by heating the solutions for 30 consecutive minutes at 60° C., after which time there was no visible precipitate. Samples were also retained within the same tubes for the duration of the study, rather than transferring supernatant fractions to fresh tubes at each time point. The concentrations were recorded eleven times; seven times within the first eight hours following the initial combination and four times over the subsequent 192 hours. The concentrations were measured at 0.5 hours, 1.0 hour, 2.0 hours, 3.0 hours, 4.0 hours, 6.0 hours, 8.0 hours, 24.0 hours, 32.0 hours, 100 hours, and 200 hours. The concentration of CN-Cbl was measured and the percentage of CN-Cbl remaining in solution was calculated.

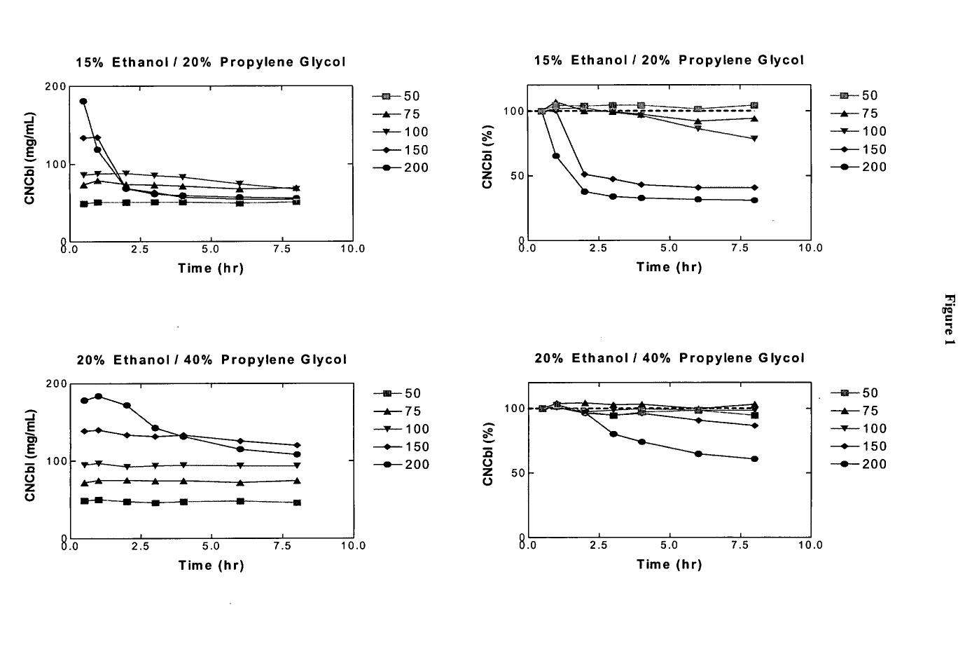

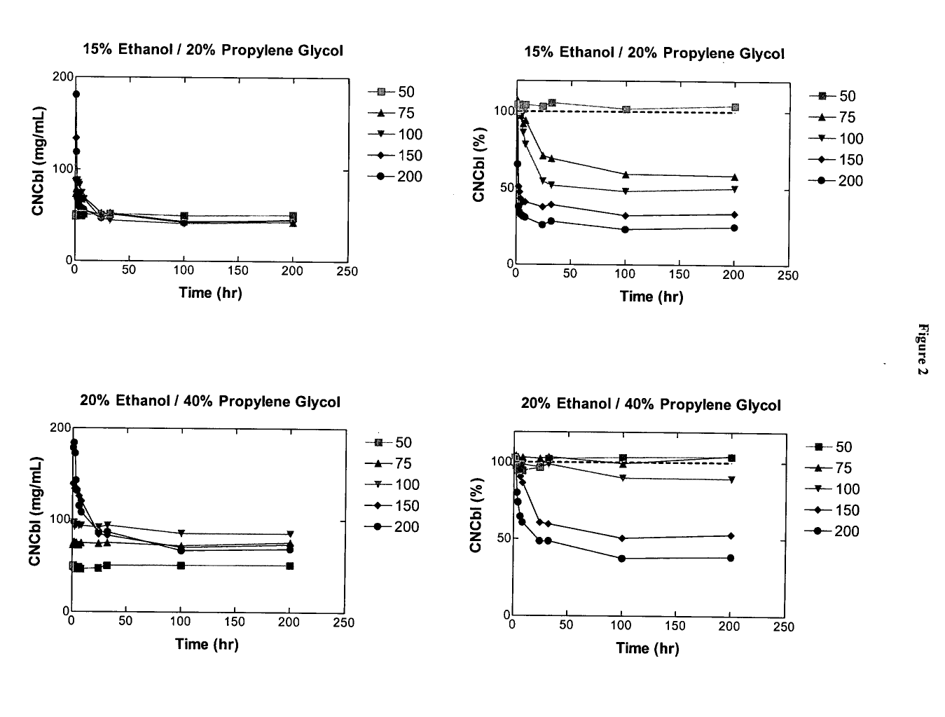

The results obtained from the 8-hour time course are shown in FIGS. 1A-1D and the entire 200-hour time course is shown in FIGS. 2A-2D. Within the first eight hours, challenge volumes of 50, 75 and 100 mg/ml CN-Cbl remained in solution in both combinations of excipients. There was some precipitation with challenge volumes of 150 and 200 mg/ml CN-Cbl, and that precipitation was greater when the CN-Cbl was dissolved in 15% ethanol and 20% propylene glycol (as opposed to 20% ethanol, 40% propylene glycol). These observations held true over the longer time course studied (see FIGS. 2A-2D).

### Example 2

### Methylcobalamin-Containing Solutions

A variety of solutions containing methylcobalamin (Me-Cbl) were tested. In one series of experiments, solutions containing ethanol, propylene glycol, or both ethanol and propylene glycol were challenged with 20 mg/ml Me-Cbl. In another series of experiments, the same solutions were challenged with 50 mg/ml Me-Cbl. The conditions were the same as those described for CN-Cbl. In both series, the excipients included: (a) 15% ethanol; (b) 20% ethanol; (c) 20% propylene glycol; (d) 40% propylene glycol; (e) 15% ethanol and 20% propylene glycol; and (f) 20% ethanol and 40% propylene glycol. Following suitable dilution, the absorbance at A340 was measured on Day 0, Day 1, and Day 2 (Table 7).

With the lower challenge (20 mg/ml Me-Cbl), the concentrations of Me-Cbl fell to below control levels after three days when the excipient contained only ethanol. When propylene glycol was used and when ethanol was used in combination with propylene glycol, the concentration of Me-Cbl remained at the challenge level. The results obtained with the higher challenge (50 mg/ml Me-Cbl) were similar, although a greater percentage of the Me-Cbl precipitated after three days. When only ethanol was used as an excipient, the concentration of Me-Cbl fell below that present in the control solution (water), but when propylene glycol or a combination of propylene glycol and ethanol were used, the concentration of Me-Cbl in solution was greater than that of the control. Where 20% ethanol and 40% propylene glycol were combined, the concentration remained very near that of the challenge amount (˜48 mg/ml) after three days.

### Example 3

### Hydroxocobalamin-Containing Solutions

A variety of solutions containing hydroxocobalamin (HO-Cbl) were tested. The excipients tested in these studies were the same as those used above: (a) 15% ethanol; (b) 20% ethanol; (c) 20% propylene glycol; (d) 40% propylene glycol; (e) 15% ethanol and 20% propylene glycol; and (f) 20% ethanol and 40% propylene glycol. Here, however, three challenge amounts were tested (˜16, 40, and 81 mg/ml HO-Cbl) and the time course was slightly longer, with measurements being made on Day 3 and Day 5. The OH-Cbl was solubilized at room temperature, as it dissolved readily in the excipients tested. If necessary or desired, the solutions can be mixed by shaking briefly (using, for example, a vortex or other agitation for a few seconds (e.g., 5-20 seconds) to a minute or so (e.g., 1-3 minutes). Following suitable dilution, the absorbance at Å351 was measured.

Unlike the other vitamin B12 compounds tested, the concentrations of HO-Cbl did not substantially exceed control values in any of the solutions tested with any of the three challenge amounts (Table 8).

### Example 4

### Cyanocobalamin Formulation

A formulation containing 60 mg/ml of Cyanocobalamin was developed containing 61.81 mg/ml choline, 40% v/v propylene glycol and 10% v/v ethanol. The formulation was buffered with 0.025 M phosphate buffer to maintain the pH at 8. The formulation was found to be physically stable at room temperature and could be diluted with water, saline or glucose solution without risk of precipitation. The formulation could not be autoclaved because of chemical degradation and could not be stored at 5° C. because it precipitated at this temperature. A proposed formulation is shown in Table 9. This formulation is suitable for further progression to clinical trial manufacture.

Materials:

Drug Substance

Cyanocobalamin (CN-Cbl) is a cobalt containing vitamin and is reported to be sparingly soluble in water in the order of 1:80 or ˜12.5 mg/ml (Merck Index 1996). The drug substance is also reported to be very hygroscopic in the anhydrous form and should be stored appropriately. The material appears as dark red crystals or as an amorphous or crystalline red powder. It has a melting point of 300° C. and a molecular weight of 1355.38. The pH of a 10 mg/ml aqueous solution is 5.02.

Excipients

The excipients investigated are summarised in Table 10.

Solubility Study

During the solubility study the following were investigated:
cosolvents (mixtures of ethanol and propylene glycol)
in-situ salt formers
surfactants
pH effects
in-situ salt formers and cosolvents
   Solubility Study Method

The method used for the preparation of the solubility samples was carried out in duplicate as follows:
A 400±5 mg quantity of CN-Cbl was weighed into a 7 ml screw top glass vial and 2 ml of the solution under test was added.
The mixture was sonicated for 5 minutes.
The samples were placed in a water bath at 25° C. with a magnetic stirrer set at 500 rpm and left to equilibrate overnight (approx. 16 hours). During the first experiment the time for the solution to equilibrate was determined and 16 hours was found to be suitable.
The sample was filtered through a 0.2 μm PVDF syringe filter.
During the later studies, the pH of the solution under test, before CN-Cbl was added, and the pH of the saturated solution were measured.
The filtrate was diluted with water and the solubility measured using a UV method. A baseline (reference cell) of water or the solution under test if this had an absorbance at 361 nm was used.
The saturated solubility was calculated from the A^{1%}_{1cm} value and the dilution of the solution.
   Results of CN-Cbl Solubility in Water and Determination of Equilibrium Time

During the measurement of the solubility of CN-Cbl in water the equilibrium time was also evaluated. The results are summarised in Table 11. These results had a mean of 15.33 mg/ml and a relative standard deviation of 0.79%. This indicates that 16 hours is suitable equilibrium time for use during the determination of the saturated solubility of CN-Cbl in various solutions.

Results of Cosolvent Solubility Experiments

The results for the solubility of CN-Cbl in mixtures of propylene glycol and ethanol are shown in Table 12.

Results of In-Situ Salt Former Solubility Experiments

The results of testing the solubility of CN-Cbl in various salt formers at a concentration of 0.1 M are shown in Table 13. During the evaluation of salt formers the pH was also measured.

Surfactant Solubility Results

The results from the evaluation of various surfactants are shown in Table 14. All of the solutions tested contained 40% v/v propylene glycol and 10% v/v ethanol as well as 5% w/v of the surfactant under test. The tested surfactants appeared to have a detrimental effect on the solubility of CN-Cbl.

Results of pH Effects

The solubility of CN-Cbl at various different pH values was investigated using buffers. The results are shown in Table 15. The best solubility result was obtained at pH 8.9, but the presence of diethanolamine in the buffer may also be acting as an in-situ salt former. There was little difference in the solubility of the other pH values tested. The solubilities obtained were markedly lower than with cosolvents or in-situ salt formers used alone.

Results of Combinations of Cosolvents and In Situ Salt Formers

Initially, the two salt formers, which had the best solubility results, were tested in combination with 40% v/v propylene glycol and 10% v/v ethanol. The salt formers were tested at a concentration of 0.1 M. The results are shown in Table 16. A level of 75 mg/ml was exceeded in the formulation containing propylene glycol, ethanol and choline. Different levels of choline in combination with 40% v/v propylene glycol, and 10% v/v ethanol were evaluated and the results shown in Table 17.

The solubility of CN-Cbl in the formulations increased slightly with an increase in the concentration of choline. All of the concentrations tested were above a target level of 75 mg/ml. The pH of these samples were high and are unlikely to be tolerated. The pH was adjusted with hydrochloric acid to various values and the solubility measured. The results of these studies are shown in Table 18.

The reduction in the pH had a significant effect on the solubility of CN-Cbl. It was difficult to adjust the pH of formulations using hydrochloric acid as the pH meter was slow to settle during readings and the pH of solutions tended to change on standing. The effect of pH on the solubility was further evaluated using buffers and the results are shown in Table 19. To obtain the required pH values buffers alone at a concentration of 0.025 M were not strong enough. Therefore hydrochloric acid was also added to the formulations. The levels of choline chosen were molar ratios with CN-Cbl. A level of 0.044 M choline is equivalent to a 1:1 molar ratio with 60 mg/ml of CN-Cbl, and 0.088 M is equivalent to a 2:1 molar ratio with 60 mg/ml CN-Cbl.

Results for Solutions Containing Choline Chloride

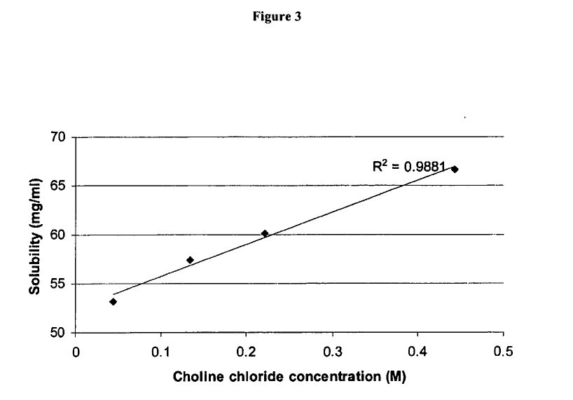

A solubility study was carried out using choline chloride in place of choline. This was used in combination with 40% v/v propylene glycol and 10% v/v ethanol. The levels of choline chloride evaluated were in molar ratios based on a formulation containing 60 mg/ml of CN-Cbl. The molecular weight of choline chloride is 139.63. The pH of the solution was adjusted to 8 using sodium hydroxide. The results of this study are shown in Table 20. The results were plotted on a graph and are shown in FIG. 3. The relationship between the concentration of choline chloride and CN-Cbl solubility is linear over the range tested. The saturated solubility of solutions buffered to pH 8 using 0.025 M phosphate buffer (7.25 mg/ml disodium hydrogen phosphate dodecahydrate and 0.74 mg/ml sodium dihydrogen phosphate dihydrate) were measured and the results are shown in Table 21. These solutions contained 40% v/v propylene glycol, 10% v/v ethanol and the level of choline chloride stated in the table. The solubility results indicated that formulation FD4068P112A containing 60 mg/ml of CN-Cbl was suitable for further evaluation. The details of this proposed formulation are shown in Table 9.

Formulation Development

Formulation of a Buffer System

During the early formulation studies it was found that the pH of CN-Cbl formulations was difficult to measure and tended to change on standing. It was also noted that changes in pH occurred during the solubility study. This indicated that the solutions require buffering to maintain the required pH. The solubility results indicated that the solubility of CN-Cbl was slightly better at high pH values compared to low pH values. A pH of 8 was thought to be the most suitable for the formulation to achieve good solubility and tolerability. A suitable 0.025 M buffer system was developed containing 7.25 mg/ml disodium hydrogen phosphate dodecahydrate and 0.74 mg/ml sodium dihydrogen phosphate dihydrate. The addition of CN-Cbl to the buffered solution reduced the pH of the solution by only 0.1 pH unit, demonstrating adequate buffering capacity.

Osmolarity

The osmolarity of the formulation could not be measured because of the high level of cosolvents resulting in a solution that did not freeze when tested with a Roebling freezing point osmometer. Therefore, the solution is effectively hypertonic and should be administered at a slow rate

Autoclave Study

An autoclave study was carried out on the proposed formulation described in Table 9. During this study the necessity for nitrogen purging and sparging was evaluated. The following four combinations were evaluated:
Not autoclaved, nitrogen purged and sparged
Autoclaved, nitrogen purged and sparged
Not autoclaved, no nitrogen
Autoclaved, no nitrogen
   Autoclave Study Method

Water for injection, which had been sparged with nitrogen, was used to prepare the solution. During the manufacture the container was regularly purged with nitrogen and the lid kept on as much as possible. 2 ml aliquots of half of the solution were filled into type 1 glass vials, which were purged with nitrogen before and after filling. The remaining solution had air bubbled through for 30 minutes before filling 2 ml aliquots into vials. Half of the vials from each set of samples were autoclaved at 121° C. for 20 minutes. All of the samples were tested for pH, assay, related substances and assessed for precipitation after 7 days at room temperature and 5° C.

Autoclave Study Results

The results of the autoclave study are summarised in Tables 22 and 23. The pH values for the samples were all close to the required value of 8, with negligible difference between the individual readings. The assay values of the samples decreased by approximately 10 mg/ml on autoclaving. The level of related substances increased on autoclaving. The increase was greater in samples that were not nitrogen purged and sparged. The results indicated that the formulation is not stable to autoclaving at these conditions. The results also indicate that some protection against oxidation can be achieved by using nitrogen purged/sparged water for injection.

Precipitate was observed in the 5° C. samples after 7 days. This was warmed to 40° C. and most of it redissolved after 4 hours. A placebo solution was prepared and stored at 5° C. After 3 weeks there was no precipitate in the placebo sample. It is recommended that CN-Cbl solutions be stored at room temperature and not stored at 5° C.

Filter Compatibility Study

A filter compatibility study was carried out on different filters to find the most suitable one for use during the manufacture of CN-Cbl solutions. The following 47 mm membranes, which had a surface area of 17.35 cm³, were assessed:
Fluorodyne® (hydrophilic polyvinylidene fluoride, PVDF) 0.2 μm from Pall
Durapore® (hydrophilic polyvinylidene fluoride, PVDF) 0.22 μm from Millipore
Supor® (hydrophilic polyethersulphone, PES) 0.2 μm from Pall
   Filter Compatibility Study Method

The formulation used in this study is described in Table 9. The following method was carried out for each filter membrane:
30 ml of the solution to be tested was placed in a 50 ml syringe.
The solution was forced through an in-line filter holder, housing the 47 mm membrane under investigation, using the syringe.
1 ml aliquots were collected at sampling points of 1, 2, 3, 4, 5, 10, 20 and 30 ml.
The samples were assayed by UV with samples of unfiltered bulk solution taken at the beginning and end of the study.
   Filter Compatibility Study Results

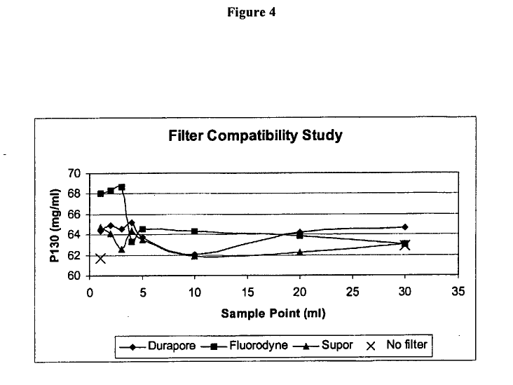

The assay results of the filter study samples are shown in Table 24. The results are also presented graphically in FIG. 4. There is no difference in the concentration of CN-Cbl in the aliquots and it can be assumed that there is no significant adsorption on any of the filters tested.

Dilution Study

A sample of the formulation described in Table 9 was diluted with the following solutions to find out if precipitation occurs:
0.9% saline
5% anhydrous glucose
Water for injection.

The results are summarised in Table 25. These results indicate that the formulation can be diluted with water, saline or glucose solution with no risk of precipitation.

Robustness of Formulation

Three batches of the formulation were prepared and the pH of each measured to ensure the formulation is robust. The results are summarised in Table 26. The pH results are consistent for the three batches indicating that the formulation is robust.

Manufacturing Process

A suitable process for the manufacture of CN-Cbl solutions was developed. The most suitable process involved the preparation of a solution without the active, then adding the CN-Cbl. By preparing a solution in this way the pH can be adjusted, if required, before the addition of the active. The active causes a slight decrease in pH, typically in the region of 0.1 units. The manufacturing process developed was as follows:
The buffer constituents were dissolved in approximately 20% of the water by stirring with a magnetic stirrer.
The choline chloride was added and stirred to dissolve.
The propylene glycol was added and the solution mixed by stirring.
The ethanol was added and stirred to mix.
The pH was checked (tentative limits pH 7.8 to pH 8.2).
The CN-Cbl was added and the mixture stirred until dissolved. An in process UV assay may be used to ensure that all of the CN-Cbl has dissolved.
   Discussion

The solubility enhancement study achieved a solubility of 53.8 mg/ml in a formulation containing 40% v/v propylene glycol and 10% v/v ethanol. The best solubility obtained using the tested salt formers was 29.5 mg/ml using choline, but the formulation had a high pH of 12.5. Surfactants had no effect on the solubility of formulations containing propylene glycol and ethanol. The solubility of CN-Cbl was slightly better at higher pH values.

The highest solubility in this study was obtained with a combination of choline, propylene glycol and ethanol. A saturated solubility of 91.8 mg/ml was obtained, but the formulation had a pH of 12.6. When the pH was reduced to a value of 8, the solubility was also reduced to 63 mg/ml. Choline chloride was found to be a better alternative to choline, as this material has a monograph listed in the United States Pharmacopoeia and gave similar results to choline during the solubility experiments. Choline chloride solutions also had a much lower pH value than choline solutions. A 0.1 M choline chloride solution containing 10% ethanol and 40% propylene glycol has a pH of approximately 7 and a 0.1 M choline solution containing the same cosolvents has a pH of approximately 12.7.

A formulation using choline chloride in a 1:10 molar ratio with CN-Cbl was identified for further evaluation. The formulation had a saturated solubility of 66.8 mg/ml for CN-Cbl. The proposed formulation has a CN-Cbl content of 60 mg/ml and also contains 0.025 M phosphate buffer to maintain the pH at approximately 8, 40% v/v propylene glycol and 10% v/v ethanol. Details of this formulation are shown in Table 9.

The formulation was found to be robust, since when three different batches were produced consistent pH values, close to 8, were obtained for all of the batches. The osmolarity of the formulation could not be measured because the high level of cosolvents render the solution hypertonic. Therefore, the solution did not freeze when tested with a Roebling freezing point osmometer.

The solution was found to be unstable when autoclaved at 121° C. for 20 minutes. This means that the solution will require aseptic preparation during manufacture. Some protection against oxidation was found to be beneficial when the solutions were purged/sparged with nitrogen.

The solution is highly coloured so an in process assay would be useful to ensure complete dissolution of the drug substance before progressing to filtration and filling. The filter study indicated that all of the filters tested, Durapore®, Fluorodyne® and Supor®, are all suitable for use during the manufacture of CN-Cbl solutions.

The solutions were physically stable when stored at room temperature for a week. A dilution study indicated that the formulation could be diluted with water, glucose or saline solutions with no risk of precipitation.

### CONCLUSIONS

A solution containing 60 mg/ml of CN-Cbl was developed suitable for clinical studies. The proposed formulation contains 0.025 M phosphate buffer, 61.81 mg/ml choline chloride, 40% v/v propylene glycol and 10% v/v ethanol. Details of the formulation are shown in Table 9.

TABLE 1
Maximum Solubility with Individual Excipients
CN-Cbl
concentrations in mg/mL

| Excipient | Propylene | | | | |
|---|---|---|---|---|---|
| % (v/v) | Glycol | Ethanol | PEG 300 | Glycerol | H₂O |
| challenge | 100 | 100 | 100 | 100 | 100 |
| | | | | | 14.1 |
| 5.0 | | | | 16.2 | |
| 10.0 | 28.1 | 29.4 | 24.1 | 18.2 | |
| 15.0 | | 35.5 | | 19.4 | |
| 20.0 | 41.8 | 52.1 | 34.4 | 21.5 | |
| 30.0 | 95.3 | | 40.5 | | |
| 40.0 | 95.1 | | 45.4 | | |
| 50.0 | 95.1 | | 45.9 | | |
| 60.0 | 95.1 | | | | |
| 70.0 | 85.3 | | | | |
| 80 | 90.1 | | | | |

TABLE 2A
Maximum Solubility with Combinations of Excipients
Propylene Glycol - Ethanol Challenge: 200

| | ETHANOL | | | | |
|---|---|---|---|---|---|
| PG | 0 | 5 | 10 | 15 | 20 |
| 0 | 17.5 | 22.5 | 28.5 | 27.7 | 176.0 |
| 20 | 31.3 | 34.4 | 55.6 | 182.5 | 177.5 |
| 30 | 35.9 | 64.8 | | 150.5 | 180.2 |
| 40 | 172.5 | 177.1 | 184.4 | 192.9 | 186.0 |
| 50 | 145.1 | 179.4 | 190.2 | 179.4 | 187.9 |
| 60 | 185.2 | 184.8 | 186.8 | 186.4 | 196.0 |

TABLE 2B
PEG 300 - Ethanol Challenge: 200

| | ETHANOL | | | | |
|---|---|---|---|---|---|
| PEG 300 | 0 | 5 | 10 | 15 | 20 |
| 0 | 23.2 | 20.2 | 37.9 | 30.0 | 50.4 |
| 10 | 36.3 | 45.2 | 61.4 | | 63.1 |
| 20 | 48.5 | 54.7 | 67.1 | 63.1 | 91.8 |
| 30 | 75.3 | 65.2 | | 79.5 | 76.2 |
| 40 | | 77.0 | 44.4 | 62.5 | 56.0 |
| 50 | 54.6 | 76.2 | | 41.5 | 40.1 |

TABLE 2C
Propylene Glycol with 20% Ethanol

PG (% v/v)

| Challenge | 0 | 10 | 10 | 30 | 40 | H₂0 |
|---|---|---|---|---|---|---|
| 50 | 52.0 | 50.9 | 52.0 | 53.4 | 51.5 | 29.2 |
| 75 | 75.6 | 76.2 | 78.1 | 80.7 | 79.9 | 22.5 |
| 100 | 101.9 | 91.8 | 101.9 | 104.4 | 104.6 | 21.2 |
| 125 | 110.0 | 121.9 | 128.9 | 130.0 | 118.1 | 21.6 |
| 150 | 81.0 | 149.0 | 149.7 | 151.3 | 152.4 | 20.1 |
| 175 | 77.2 | 161.7 | 177.1 | 168.6 | 159.0 | 21.6 |
| 200 | 64.8 | 148.2 | 193.7 | 196.8 | 192.2 | 19.8 |

TABLE 2D
Propylene Glycol - Ethanol Challenge: 400

| | ETHANOL | | | | |
|---|---|---|---|---|---|
| PG | 0 | 5 | 10 | 15 | 20 |
| 0 | 21.5 | 27.4 | 33.5 | 39.3 | 44.7 |
| 20 | 37.2 | 43.0 | 53.6 | 68.7 | 85.7 |
| 30 | 47.2 | 58.3 | 68.7 | 97.6 | 98.0 |
| 40 | 62.5 | 85.9 | 98.0 | 104.6 | 114.8 |

TABLE 3A
After 3 days at Room Temperature

| | PG (% v/v) | | | | | | |
|---|---|---|---|---|---|---|---|
| challenge | 0 | 10 | 20 | 30 | 40 | 50 | 60 |
| 50 | 13.6 | 19.5 | 25.3 | 31.0 | 50.0 | 49.9 | 51.5 |
| 75 | 15.0 | 20.1 | 25.8 | 30.5 | 40.5 | 67.0 | 75.8 |
| 100 | 14.9 | 19.5 | 25.0 | 28.8 | 37.6 | 55.9 | 97.2 |
| 125 | 14.5 | 20.4 | 23.1 | 28.3 | 38.6 | 46.2 | 78.7 |

TABLE 3B

| | EtOH (% v/v) | | | | |
|---|---|---|---|---|---|
| challenge | 0 | 10 | 20 | 30 | 40 |
| 50 | 13.6 | 20.5 | 26.9 | 40.0 | 47.6 |
| 75 | 15.0 | 23.0 | 26.1 | 43.0 | 53.4 |
| 100 | 14.9 | 24.0 | 27.8 | 34.5 | 46.1 |
| 125 | 14.5 | 21.5 | 28.4 | 37.9 | 50.9 |

TABLE 4
Maximum Solubility in Solution of Combination of Excipients
CN-Cbl
After 2 weeks at Room Temperature

| Challenge | | Propylene | |
|---|---|---|---|
| CN-Cbl | Ethanol | Glycol | Conc |
| mg/mL | % (v/v) | % (v/v) | (mg/mL) |
| 50 | 0 | 0 | 11.46 |
| | 10 | 20 | 26.68 |
| | 10 | 30 | 39.46 |
| | 10 | 40 | 50.65 |
| | 15 | 20 | 48.72 |
| | 15 | 30 | 49.78 |
| | 15 | 40 | 49.39 |
| | 20 | 20 | 47.08 |
| | 20 | 30 | 49.78 |
| | 20 | 40 | 49.87 |
| 75 | 0 | 0 | 12.31 |
| | 10 | 20 | 26.91 |
| | 10 | 30 | 35.21 |
| | 10 | 40 | 55.66 |
| | 15 | 20 | 33.28 |
| | 15 | 30 | 43.31 |
| | 15 | 40 | 74.28 |
| | 20 | 20 | 38.39 |
| | 20 | 30 | 59.62 |
| | 20 | 40 | 75.82 |

TABLE 5
Effects of Temperature of Heating Solution

| Time at Room Temperature | | | 60 degrees | |
|---|---|---|---|---|
| Challenge | | Propylene | After 3 days | |
| CN-Cbl | Ethanol | Glycol | Conc | 37 degrees |
| mg/mL | % (v/v) | % (v/v) | (mg/mL) | After 2 weeks |
| 50 | 0 | 0 | 12.85 | |
| | 20 | 0 | 24.41 | |
| | 20 | 10 | 32.32 | |
| | 20 | 20 | 49.87 | 47.08 |
| | 20 | 30 | 48.33 | 49.78 |
| | 20 | 40 | 50.36 | 49.87 |
| 75 | 0 | 0 | 14.01 | |
| | 20 | 0 | 24.41 | |
| | 20 | 10 | 30.10 | |
| | 20 | 20 | 60.78 | 38.39 |
| | 20 | 30 | 76.60 | 59.62 |
| | 20 | 40 | 74.28 | 75.82 |

TABLE 6
Effect of Reheating Samples

| | Solution A | Solution B | Solution C |
|---|---|---|---|
| | (mg/ml) | (mg/ml) | (mg/ml) |
| Day 1 | 94.73 | 141.62 | 185.22 |
| Day 4 no reheating | 43.80 | 71.39 | 59.42 |
| Day 4 reheating to 60° C. | 94.73 | 141.23 | 169.78 |
| Day 4 reheating to 50° C. | 83.54 | 124.25 | 133.13 |

TABLE 7

| Me-Cbl | | | | | | | | | |
|---|---|---|---|---|---|---|---|---|---|
| Challenge | | Propylene | | | | | Day 0 | Day 1 | Day 3 |
| Me-Cbl | Ethanol | Glycol | | | | Conc | Conc | Conc | Conc |
| mg/mL | % (v/v) | % (v/v) | Dilution | A340 | Atot | (mM) | (mg/mL) | (mg/mL) | (mg/mL) |
| 20 | 0 | 0 | 400 | 0.425 | 170.0 | 12.78 | 17.18 | 14.03 | 13.18 |
| | 15 | 0 | 800 | 0.282 | 225.6 | 16.96 | 22.80 | 12.53 | 11.12 |
| | 20 | 0 | 800 | 0.286 | 228.8 | 17.20 | 23.13 | 9.18 | 9.18 |
| | 0 | 20 | 800 | 0.292 | 233.6 | 17.56 | 23.61 | 24.02 | 23.37 |
| | 0 | 40 | 800 | 0.296 | 236.8 | 17.80 | 23.94 | 24.34 | 24.50 |
| | 15 | 20 | 800 | 0.281 | 224.8 | 16.90 | 22.72 | 23.45 | 21.91 |
| | 20 | 40 | 800 | 0.282 | 225.6 | 16.96 | 22.80 | 23.86 | 24.10 |
| 50 | 0 | 0 | 400 | 0.313 | 125.2 | 9.41 | 12.66 | 12.86 | 12.86 |
| | 15 | 0 | 2000 | 0.280 | 560.0 | 42.11 | 56.61 | 11.93 | 11.32 |
| | 20 | 0 | 2000 | 0.287 | 574.0 | 43.16 | 58.02 | 10.47 | 10.35 |
| | 0 | 20 | 2000 | 0.288 | 576.0 | 43.31 | 58.22 | 17.99 | 17.79 |
| | 0 | 40 | 2000 | 0.283 | 566.0 | 42.56 | 57.21 | 23.21 | 22.16 |
| | 15 | 20 | 2000 | 0.280 | 560.0 | 42.11 | 56.61 | 15.49 | 15.45 |
| | 20 | 40 | 2000 | 0.285 | 570.0 | 42.86 | 57.62 | 51.15 | 48.12 |

TABLE 8

| Chal- | | | | | |
|---|---|---|---|---|---|
| lenge | | Propylene | Conc | Conc | Conc |
| OH-Cbl | Ethanol | Glycol % | (mg/mL) | (mg/mL) | (mg/mL) |
| mg/mL | % (v/v) | (v/v) | Day 0 | Day 3 | Day 5 |
| 16.18 | 0 | 0 | 0.00 | 16.83 | 16.93 |
| | 15 | 0 | 0.00 | 16.67 | 17.06 |
| | 20 | 0 | 0.00 | 17.05 | 17.14 |
| | 0 | 20 | 0.00 | 17.10 | 17.31 |
| | 0 | 40 | 0.00 | 16.78 | 17.10 |
| | 15 | 20 | 0.00 | 16.94 | 17.44 |
| | 20 | 40 | 0.00 | 16.25 | 16.59 |
| 40.45 | 0 | 0 | 0.00 | 41.90 | 41.68 |
| | 15 | 0 | 0.00 | 41.15 | 41.47 |
| | 20 | 0 | 0.00 | 41.58 | 41.04 |
| | 0 | 20 | 0.00 | 41.68 | 41.47 |
| | 0 | 40 | 0.00 | 41.79 | 41.79 |
| | 15 | 20 | 0.00 | 41.04 | 42.22 |
| | 20 | 40 | 0.00 | 41.36 | 41.58 |
| 80.90 | 0 | 0 | 0.00 | 81.02 | 80.80 |
| | 15 | 0 | 0.00 | 82.73 | 82.94 |
| | 20 | 0 | 0.00 | 82.09 | 80.59 |
| | 0 | 20 | 0.00 | 83.80 | 82.09 |
| | 0 | 40 | 0.00 | 81.87 | 82.73 |
| | 15 | 20 | 0.00 | 81.23 | 80.16 |
| | 20 | 40 | 0.00 | 82.94 | 82.73 |

TABLE 9
Proposed Formulation

| | | |
|---|---|---|
| Material | Quantity per ml | % w/v |

| | | | |
|---|---|---|---|
| Cyanocobalamin EP, USP, JP | 60.00 | mg | 6.000 |
| Disodium hydrogen phosphate | 7.25 | mg | 0.725 |
| dodecahydrate EP, USP | | | |
| Sodium dihydrogen phosphate | 0.74 | mg | 0.074 |
| dehydrate EP, USP | | | |
| Propylene glycol EP, USP | 0.40 | ml | 0.040 |
| Ethanol EP, USP | 0.10 | ml | 0.010 |
| Choline chloride USP | 61.81 | mg | 6.181 |
| Water for injection EP, USP | q.s. to 1.00 | ml | q.s. to 100 |

Molar ratio of Cyanocobalamin to choline chloride 1:10

TABLE 10
Excipients

| Excipient | Function | Supplier |
|---|---|---|
| Disodium hydrogen phosphate | Buffer | Merck |
| dodecahydrate | | |
| Sodium dihydrogen phosphate | Buffer | Merck |
| dehydrate | | |
| Choline chloride | In-situ salt former | BDH |
| Propylene glycol | Cosolvent | BDH |
| Ethanol | Cosolvent | Hayman |
| Water for injection | Solvent | Patheon |
| Sodium hydroxide | pH adjuster | Merck |
| Hydrochloric acid (2 M) | pH adjuster | VWR |
| Citric acid | Buffer | SA Citrique |
| Sodium citrate | Buffer | SA Citrique |
| Polysorbate 20 | Surfactant | Croda |
| Polysorbate 80 | Surfactant | Univar |
| Poloxamer 188 | Surfactant | Ellis & Everard |
| Cremaphor RH40 | Surfactant | BASF |
| Choline | In-situ salt former | Sigma |
| Diethanolamine | In-situ salt former | Sigma |
| Triethylamine | In-situ salt former | Sigma |
| Triethanolamine | In-situ salt former | Fluka |
| L-arginine | In-situ salt former | Sigma |
| Meglumine | In-situ salt former | Sigma |
| L-lysine | In-situ salt former | Sigma |

TABLE 11
Solubility of CN-Cbl in Water

| Equilibrium Time (hours) | Saturated Solubility (mg/ml) |
|---|---|
| 16 | 15.25 |
| 24 | 15.47 |
| 40 | 15.27 |

TABLE 12
Solubility of CN-Cbl in Propylene Glycol Ethanol Mixtures

| Ref. | Propylene glycol | Ethanol | Saturated Solubility |
|---|---|---|---|
| FD4068 | (% v/v) | (% v/v) | (mg/ml) |
| 14A | 30 | 5 | 36.6 |
| 15A | 32 | 5 | 39.8 |
| 15B | 34 | 5 | 39.1 |
| 15C | 36 | 5 | 41.8 |
| 16A | 38 | 5 | 45.5 |
| 16B | 40 | 5 | 48.1 |
| 16C | 30 | 10 | 45.6 |
| 16D | 32 | 10 | 45.8 |
| 17A | 34 | 10 | 47.8 |
| 17B | 36 | 10 | 51.0 |
| 17C | 38 | 10 | 52.2 |
| 17D | 40 | 10 | 53.8 |

TABLE 13
Solubility of CN-Cbl in In-Situ Salt Formers

| | | | pH of | Saturated |
|---|---|---|---|---|
| | Concentration | Initial | CN-Cbl | Solubility |
| Salt | (mg/ml) | pH | Solution | (mg/ml) |
| Choline | 12.12 | 12.54 | 12.49 | 29.5 |
| Triethylamine | 10.12 | 11.64 | 11.42 | 24.9 |
| Triethanolamine | 14.92 | 10.13 | 9.26 | 18.6 |
| L-arginine | 17.42 | 10.88 | 10.22 | 18.2 |
| Diethanolamine | 10.51 | 10.80 | 10.15 | 18.1 |
| Meglumine | 19.52 | 11.00 | 10.70 | 18.0 |
| L-lysine | 16.42 | 9.60 | 9.59 | 16.3 |

TABLE 14
Solubility of CN-Cbl in Surfactants

| Ref.: FD4068 | Surfactant | Saturated Solubility (mg/ml) |
|---|---|---|
| 17D | None | 53.8 |
| 26A | Polysorbate 80 | 49.0 |
| 26B | Polysorbate 20 | 46.5 |
| 26C | Paloxamer 188 | 45.1 |
| 27A | Cremophor RH40 | 47.1 |

TABLE 15
pH Effects

| | | | pH of | Saturated |
|---|---|---|---|---|
| Ref.: | | Initial | CN-Cbl | Solubility |
| FD4068 | Buffer Constituents | pH | Solution | (mg/ml) |
| 29A | Sodium citrate, citric acid | 2.64 | 2.89 | 15.3 |
| 30A | Sodium citrate, citric acid | 4.75 | 4.83 | 15.0 |
| 30B | Disodium hydrogen phosphate, | 7.03 | 7.02 | 13.6 |
| | sodium dihydrogen phosphate | | | |
| 31A | Diethanolamine, hydrochloric | 8.93 | 8.80 | 17.5 |
| | acid | | | |

TABLE 16
Results of Cosolvents and Salt Former Combinations

| Ref.: | | | pH of CN-Cbl | Saturated |
|---|---|---|---|---|
| FD4068 | Salt | Initial pH | solution | Solubility (m/ml) |
| 33 | Choline | 12.68 | 12.61 | 91.8 |
| 34 | Triethylamine | 11.47 | 10.97 | 65.1 |

TABLE 17
Results for Different Levels of Choline

| | | | | | Saturated | Saturated |
|---|---|---|---|---|---|---|
| | | | | pH of | Solu- | Solu- |
| Ref.: | Choline | Choline | Initial | CN-Cbl | bility | bility |
| FD4068 | (M) | (mg/ml) | pH | Solultion | (mg/ml) | (M) |
| 42A | 0.055 | 6.71 | 12.49 | 12.44 | 80.8 | 0.060 |
| 42B | 0.068 | 8.23 | 12.63 | 12.53 | 82.8 | 0.061 |
| 33 | 0.100 | 12.12 | 12.68 | 12.61 | 91.8 | 0.068 |
| 43A | 0.111 | 13.41 | 12.80 | 12.76 | 92.1 | 0.068 |

TABLE 18
Solubility Results for pH Adjusted Solutions Containing 10%
v/v Ethanol, 40% v/v Propylene Glycol and 0.1 M Choline

| | | | Saturated |
|---|---|---|---|
| | Initial | pH of CN-Cbl | Solubility |
| Ref.: FD4068 | pH | Solution | (mg/ml) |
| 39C | 7.43 | 8.00 | 63.0 |
| 38B | 7.85 | 7.70 | 59.4 |
| 38A | 8.58 | 8.10 | 60.3 |
| 39B | 8.99 | 8.01 | 63.1 |

TABLE 19
Solubility Results for Various Buffered Formulations

| Ref.: | | Choline | Choline | Initial | pH of CN-Cbl | Saturated |
|---|---|---|---|---|---|---|
| FD4068 | Buffer | (M) | (mg/ml) | pH | Solution | Solubility (mg/ml) |
| 89B | 0.15 ml 2m HCl + 0.025M | 0.044 | 5.34 | 7.03 | 6.97 | 52.6 |
| | pH 5.6 citrate buffer | | | | | |
| 96 | 0.2 ml 2M HCl + 0.025M | 0.044 | 5.34 | 8.10 | 7.96 | 54.5 |
| | pH 7.0 phosphate buffer | | | | | |
| 102A | 0.4m 2M HCl + 0.025M | 0.088 | 10.67 | 8.12 | 7.97 | 58.5 |
| | pH 6.8 phosphate buffer | | | | | |
| 103 | None - pH adjusted with | 0.044 | 5.34 | 8.05 | 7.21 | 54.6 |
| | HCl | | | | | |

TABLE 20
Solubility Results for Formulations Containing Choline Chloride

| | | | Choline | Initial pH | pH of | pH of | |
|---|---|---|---|---|---|---|---|
| Ref.: | Molar | Choline | chloride | of placebo | placebo | CN-Cbl | Saturated |
| FD4068 | Ratio | chloride (M) | (mg/ml) | solution | after 16 h | solution | solubility (mg/ml) |
| 107A | 1:1 | 0.044 | 6.14 | 7.97 | 7.70 | 6.36 | 53.2 |
| 109A | 1:3 | 0.133 | 18.57 | 7.94 | 7.50 | 6.63 | 57.4 |
| 108B | 1:5 | 0.221 | 30.86 | 7.91 | 7.39 | 6.51 | 60.1 |
| 110A | 1:10 | 0.443 | 61.81 | 7.92 | 7.80 | 6.40 | 66.6 |

TABLE 21
Solubility Results for Buffered Formulations

| | | | Choline | Initial pH | pH of | pH of | |
|---|---|---|---|---|---|---|---|
| Ref.: | Molar | Choline | chloride | of placebo | placebo | CN-Cbl | Saturated |
| FD4068 | Ratio | chloride (M) | (mg/ml) | solution | after 16 h | solution | solubility (mg/ml) |
| 112A | 1:10 | 0.443 | 61.81 | 8.08 | 8.11 | 7.93 | 66.8 |
| | based on | | | | | | |
| | 60 mg/ml | | | | | | |
| | CN-Cbl | | | | | | |
| 112B | 1:5 based | 0.184 | 25.75 | 8.14 | 8.14 | 7.97 | 62.7 |
| | on | | | | | | |
| | 50 mg/mi | | | | | | |
| | CN-Cbl | | | | | | |

TABLE 22
Autoclave Study - pH and Assay Results

| | | | | Related | |
|---|---|---|---|---|---|
| Ref.: | Auto- | | | Substances* | Assay |
| FD4068 | claved | Nitrogen | pH | (% area) | (mg/ml) |
| 119A | No | Yes | 8.04 | 2.93 | 65.4 |
| 119B | Yes | Yes | 7.95 | 25.55 | 56.8 |
| 119C | No | No | 7.98 | 3.10 | 67.1 |
| 119D | Yes | No | 8.01 | 42.87 | 55.9 |

*The level of related substances quoted on the certificate of analysis for the active was 2.8%.

TABLE 23
Autoclave Study - Physical Stability

| Ref.: | | | Precipitate Present after 7 days | |
|---|---|---|---|---|
| FD4068 | Autoclaved | Nitrogen | 5° C. | Room Temperature |
| 119A | No | Yes | Yes | No |
| 119B | Yes | Yes | No | No |
| 119C | No | No | Yes | No |
| 119D | Yes | No | No | No |

TABLE 24
Filter Study Results
Assay of Filtrate Samples (mg/ml)

| | Sampling Points | | | | | | | |
|---|---|---|---|---|---|---|---|---|
| Membranes | 1 ml | 2 ml | 3 ml | 4 ml | 5 ml | 10 ml | 20 ml | 30 ml |
| Durapore ® | 64.37 | 64.88 | 64.52 | 65.22 | 63.73 | 62.06 | 64.17 | 64.67 |
| Fluorodyne ® | 68.02 | 68.27 | 68.68 | 63.28 | 64.53 | 64.33 | 63.82 | 63.01 |
| Supor ® | 64.77 | 64.14 | 62.58 | 64.39 | 63.46 | 61.86 | 62.25 | 63.07 |

Assay of Non-Filtered Stock Solution (mg/ml)

Sampling Points

| | Start | End |
|---|---|---|
| No filter | 61.69 | 62.82 |

TABLE 25
Dilution Study Results

Precipitate Present

| Diluent | Initial | 1 hour | 2 hours | 24 hours |
|---|---|---|---|---|
| Water | No | No | No | No |
| Saline | No | No | No | No |
| Glucose | No | No | No | No |

TABLE 26
Comparison of pH Values of Three Different Batches

| Reference | pH Value |
|---|---|
| FD4068P119 | 7.98 |
| FD4068P123B | 8.05 |
| FD4068P 131A | 8.08 |

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Thus, the present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the compounds, methodologies etc. which are reported therein which might be used in connection with the invention. Nothing herein is to be construed as an admission that the invention is not entitled to antedate such disclosure by virtue of prior invention.

What is claimed is:

1. A pharmaceutical composition comprising a vitamin B12 compound and one or more excipient that substantially solubilizes the vitamin B12 compound.

2. A pharmaceutical composition of claim 1 wherein the excipient essentially completely solubilizes the vitamin B12 compound.

3. A pharmaceutically acceptable composition, which is a solution, comprising a vitamin B12 compound and at least one alcohol, wherein the composition contains at least about 20 mg/ml of the vitamin B12 compound.

4. A composition of any preceding claim wherein the excipient provides an increase in solubility of the vitamin B12 compound of at least about 2-20 fold, more particularly 2, 5, 10, or 15 fold compared with the solubility of the vitamin B12 compound in water.

5. A pharmaceutically acceptable composition, which is a solution, comprising a vitamin B12 compound and at least one excipient, wherein the composition contains at least about 20 mg/ml of the vitamin B12 compound and the excipient is ethanol, propylene glycol, a polyethylene glycol (PEG), glycerol, mannitol, sorbitol, Tween 20, or dimethylsulfoxide or a combination thereof.

6. A pharmaceutically acceptable composition, which is a solution, consisting of a vitamin B12 compound, water, and an excipient, wherein the composition contains at least about 20 mg/ml of the vitamin B12 compound.

7. A composition of claim 3, wherein the at least one alcohol is ethanol, propylene glycol, a polyethylene glycol (PEG), a glycerol, sorbitol, or mannitol or a combination thereof.

8. A composition of claim 7, further comprising Tween 20 or dimethylsulfoxide.

9. A composition of claim 7, wherein the polyethylene glycol is PEG 200 or PEG 300.

10. A composition of claim 3, 7, 8, or 9, wherein 2-5%, 5-10%, 2-10%, 10-20%, 20-30%, 30-40%, 40-50%, 50-60%, 60-70% or 70-80% of the composition, by volume, is the alcohol.

11. A composition of claim 3 or 7, wherein the composition comprises 5-10, 10-15, or 15-20% ethanol and 20-30, 30-40, 40-50, or 50-60% propylene glycol or 10-20, 20-30, 30-40, or 40-50% PEG.

12. A composition of any preceding claim wherein the concentration of vitamin B12 compound is at least about 30-500 mg/ml, more particularly 60-200 mg/ml.

13. A composition of any preceding claim, wherein the composition comprises 5-10, 10-15, or 15-20% ethanol and 60% propylene glycol.

14. A composition of any preceding claim comprising a salt former.

15. A composition of claim 14 wherein the salt former is an organic base.

16. A composition of claim 15 wherein the organic base is choline or choline chloride.

17. A composition of claim 15 or 16 wherein the molar ratio of the organic base to vitamin B12 compound is about 1:1 to about 1:15.

18. A composition of any preceding claim, wherein the vitamin B12 compound is cyanocobalamin.

19. A composition of any preceding claim, wherein the vitamin B12 compound is adenosylcobalamin, aquocobalamin, hydroxocobalamin, methylcobalamin, or 5-o-methylbenzylcobalamin.

20. A composition of any preceding claim, wherein the vitamin B12 compound is an analog or derivative of adenosylcobalamin, aquocobalamin, cyanocobalamin, hydroxocobalamin, methylcobalamin, or 5-o-methylbenzylcobalamin.

21. A composition of claim 20, wherein the analog is a desdimethyl, monoethylamide, or methylamide analogue.

22. A composition of any preceding claim, wherein the concentration of the vitamin B12 compound is at least about 25, 30, 35, 40, 45, 50, 55, 60, 65, 70, 75, 80, 85, 90, 95, 100, 125, 150, 175, 200, 225, 250, 275 or 300 mg/ml.

23. A composition of claim 1 comprising (a) 50-200 mg/ml cyanocobalamin, 15% or 20% ethanol, and 20% or 40% propylene glycol; (b) 50-200 mg/ml, more particularly 50-100 mg/ml, cyanocobalamin, 15% ethanol, and 20% propylene glycol; (c) 50-200 mg/ml, more particularly 50-180 mg/ml cyanocobalamin, 20% ethanol, and 40% propylene glycol; (d) 50-200 mg/ml, more particularly 150-195 mg/ml cyanocobalamin, 20% ethanol, and 40% propylene glycol; (e) 50-200 mg/ml, more particularly 50-180 mg/ml cyanocobalamin, 20% ethanol, and 30% propylene glycol; or (f) 50-200, 50-100, 60-100, or 60-80 mg/ml of cyanocobalamin, 5-100 mg/ml, 5-70 mg/ml, 5-50 mg/ml, 5-25 mg/ml, or 5-20 mg/ml choline, 0-60%, 20-40%, 20%, 30%, or 40% v/v propylene glycol and 10-25%, 10-20% %, 10%, 15%, or 20% v/v ethanol.

24. A composition of any preceding claim, wherein the composition is formulated for parenteral administration.

25. A composition of any preceding claim, wherein the composition is formulated for intramuscular, intravenous, or subcutaneous administration.

26. A composition of any preceding claim, further comprising an anti-proliferative, anti-inflammatory, or anti-viral agent.

27. A composition of claim 26, wherein the anti-proliferative agent is an anti-neoplastic agent.

28. A composition of claim 27, wherein the anti-neoplastic agent is an interferon.

29. A composition of claim 28, wherein the interferon is interferon-alpha or interferon-beta.

30. A method of making a composition, which is a solution, comprising a vitamin B12 compound and at least one excipient, wherein the vitamin B12 compound is present at a concentration of at least about 20 mg/ml, the method comprising:
(a) providing an amount of the vitamin B12 compound;
(b) providing an aqueous solution comprising at least one excipient;
(c) generating a mixture of the vitamin B12 compound and the excipient, the volume of the solution being such that the concentration of the vitamin B12 compound in the mixture will be at least about 20 mg/ml;
(d) shaking the mixture; and
(e) heating the mixture.

31. A method of claim 30, wherein steps (d) and (e) are repeated until the vitamin B12 compound is solubilized in the solution comprising the excipient.

32. The method of claim 30 or 31, wherein the shaking comprises vortexing the mixture for about 15-90 seconds and the heating occurs at 37-65° C.

33. A method of treating a patient who has a vitamin B12 deficiency, the method comprising administering to the patient a therapeutically effective amount of a composition of any preceding claim.

34. A method of treating a patient who has an anti-proliferative disorder, an inflammatory disease, or a viral infection, the method comprising administering to the patient a therapeutically effective amount of a composition of any preceding claim.

35. A method of claim 33 or 34, wherein the composition is heated prior to administration.

36. Use of a composition of any preceding claim in the preparation of a medicament to treat and/or prevent a vitamin B12 deficiency disease, a proliferative disease, inflammatory disease, and/or a viral disease.
